(19) United States
(12) Patent Application Publication
(10) Pub. No.: US 2008/0090915 A1
(43) Pub. Date: Apr. 17, 2008

(54) METHOD FOR PREVENTING OR ATTENUATING ANTHRACYCLINE-INDUCED CARDIOTOXICITY

(75) Inventors: Moussa B.H. Youdim, Haifa (IL); Ofer Binah, Nofit (IL); Zaid A. Abassi, Haifa (IL); Yaron Barac, KIryat Motzkin (IL)

Correspondence Address: BROWDY AND NEIMARK, P.L.L.C.;624 NINTH STREET, NW, SUITE 300, WASHINGTON, DC, 20001-5303, US

(73) Assignees: Technion Research and Development Foundation Ltd., Hafia (IL); Rappaport Family Institute for Research in the Medical Sciences, Hafia (IL)

(21) Appl. No.: 11/874,788

(22) Filed: Oct. 18, 2007

Related U.S. Application Data
(63) Continuation-in-part of application No. 11/449,862, filed on Jun. 9, 2006.
Application No. 11/449,862 is a continuation-in-part of application No. 10/952,367, filed on Sep. 29, 2004.
(60) Provisional application No. 60/524,616, filed on Nov. 25, 2003.
(60) Provisional application No. 60/570,496, filed on May 13, 2004.

(51) Int. Cl. A61K 31/137; A61P 9/00

(52) U.S. Cl. 514/647

(57) ABSTRACT

Propargylamine, propargylamine derivatives including N-propargyl-1-aminoindan, enantiomers and analogs thereof, and pharmaceutically acceptable salts thereof, are useful for prevention or attenuation of anthracycline-induced cardiotoxicity.

### CROSS-REFERENCE TO RELATED APPLICATIONS

The present application is a continuation-in-part application of U.S. patent application Ser. No. 11/449,862, filed Jun. 9, 2006, which is a continuation-in-part application of U.S. patent application Ser. No. 10/952,367, filed Sep. 29, 2004, and claims the benefit of U.S. Provisional Patent Application No. 60/524,616, filed Nov. 25, 2003, now expired, and U.S. Provisional Patent Application No. 60/570,496, filed May 13, 2004, now expired, the entire contents of each and all these applications being herewith incorporated by reference in their entirety as if fully disclosed herein.

### FIELD OF THE INVENTION

The present invention relates to a method for preventing or attenuating anthracycline-induced cardiotoxicity and, more particularly, to propargylamine and derivatives thereof for use in said method.

### BACKGROUND OF THE INVENTION

Doxorubicin or adriamycin is a quinine-containing anthracycline and is the most widely prescribed and effective chemotherapeutic agent utilized in oncology. All anthracyclines contain a common quinone moiety, readily participating in oxidation-reduction reactions that ultimately generate highly reactive oxygen species thought to be responsible for anthracycline-induced cardiomyopathy (Sarvazyan, 1996). Doxorubicin is indicated in a wide range of human malignancies, including tumors of the bladder, stomach, ovary, lung and thyroid, and is one of the most active agents available for treatment of breast cancer and other indications, including acute lymphoblastic and myelogenous leukemias, Hodgkin's and non-Hodgkin's lymphomas, Ewing's and osteogenic bone tumors, soft tissue sarcomas, and pediatric cancers such as neuroblastoma and Wilms' tumors (Doroshow, 2001). However, the utility of doxorubicin is limited by cumulative, dose-related, potentially fatal, progressive and often irreversible cardiac toxicity that may lead to congestive heart failure (Swain et al., 2003).

Anthracycline cardiotoxicity may be either acute or chronic. Acute effects include electrocardiographic changes such as sinus tachycardia, ectopic contractions, T-wave changes, decreased QRS voltage, prolonged Q-T intervals and heart block. These acute toxicities are generally reversible and clinically insignificant, and do not predict future cumulative drug-related cardiac complications. In contrast, chronic anthracycline-induced cardiotoxicity is characterized by myocardial dysfunction and congestive heart failure, most often starting after one year of treatment. Chronic effects are typically irreversible and associated with cumulative drug exposure. Nevertheless, and despite these side effects, the benefits of anti-cancerous therapies including anthracycline chemotherapeutic agents such as doxorubicin, outweighs the risks, so that drugs aimed at minimizing cardiomyocytes damage are actively sought.

Propargylamine and Propargylamine Derivatives

Several propargylamine derivatives have been shown to selectively inhibit monoamine oxidase (MAO)-B and/or MAO-A activity, and, thus to be suitable for treatment of neurodegenerative diseases such as Parkinson's and Alzheimer's disease. In addition, these compounds have been further shown to protect against neurodegeneration by preventing apoptosis.

Rasagiline, R(+)-N-propargyl-1-aminoindan, a highly potent selective irreversible monoamine oxidase (MAO)-B inhibitor, has been shown to exhibit neuroprotective activity and antiapoptotic effects against a variety of insults in cell cultures and in vivo.

Rasagiline has been recently approved for treatment of Parkinson's disease in Europe, Israel, and in the U.S., under the name AZILECT® or AGILECT®, (Teva Pharmaceutical Industries Ltd., Israel). The drug is effective with a dose as low as 1 mg/kg in monotherapy and as an adjunct to L-dopa, comparable in its effect to the anti-Parkinson catechol-O-methyltransferase (COMT) inhibitor, entacapone (Brooks and Sagar, 2003).

Rasagiline exhibits neuroprotective activities both in vitro and in vivo (for review see Mandel et al., 2003; Youdim, 2003) which may contribute to its possible disease modifying activity. It is metabolized to its major two metabolites: aminoindan and S(−)-N-propargyl-1-aminoindan (here designated “TVP1022”) (Youdim et al., 2001a), which also have neuroprotective activity against serum deprivation and 1-methamphetamine-induced neurotoxicity in partially differentiated PC-12 cells (Am et al., 2004).

Rasagiline [R(+)-N-propargyl-1-aminoindan] and pharmaceutically acceptable salts thereof were first disclosed in U.S. Pat. No. 5,387,612, U.S. Pat. No. 5,453,446, U.S. Pat. No. 5,457,133, U.S. Pat. No. 5,576,353, U.S. Pat. No. 5,668,181, U.S. Pat. No. 5,786,390, U.S. Pat. No. 5,891,923, and U.S. Pat. No. 6,630,514 as useful for the treatment of Parkinson's disease, memory disorders, dementia of the Alzheimer type, depression, and the hyperactive syndrome. The 4-fluoro-, 5-fluoro- and 6-fluoro-N-propargyl-1-aminoindan derivatives were disclosed in U.S. Pat. No. 5,486,541 for the same purposes.

U.S. Pat. No. 5,519,061, U.S. Pat. No. 5,532,415, U.S. Pat. No. 5,599,991, U.S. Pat. No. 5,744,500, U.S. Pat. No. 6,277,886. U.S. Pat. No. 6,316,504, U.S. Pat. No. 5,576,353, U.S. Pat. No. 5,668,181, U.S. Pat. No. 5,786,390, U.S. Pat. No. 5,891,923, and U.S. Pat. No. 6,630,514 disclose R(+)-N-propargyl-1-aminoindan and pharmaceutically acceptable salts thereof as useful for treatment of additional indications, namely, an affective illness, a neurological hypoxia or anoxia, neurodegenerative diseases, a neurotoxic injury, stroke, brain ischemia, a head trauma injury, a spinal trauma injury, schizophrenia, an attention deficit disorder, multiple sclerosis, and withdrawal symptoms.

U.S. Pat. No. 6,251,938 describes N-propargyl-phenylethylamine compounds, and U.S. Pat. No. 6,303,650, U.S. Pat. No. 6,462,222 and U.S. Pat. No. 6,538,025 describe N-propargyl-1-aminoindan and N-propargyl-1-aminotetralin compounds, said to be useful for treatment of depression, attention deficit disorder, attention deficit and hyperactivity disorder, Tourette's syndrome, Alzheimer's disease and other dementia such as senile dementia, dementia of the Parkinson's type, vascular dementia and Lewy body dementia.

The first compound found to selectively inhibit MAO-B was R-(−)-N-methyl-N-(prop-2-ynyl)-2-aminophenylpropane, also known as L-(−)-deprenyl, R-(−)-deprenyl, or selegiline. In addition to Parkinson's disease, other diseases and conditions for which selegiline is disclosed as being useful include: drug withdrawal (WO 92/21333, including withdrawal from psychostimulants, opiates, narcotics, and barbiturates); depression (U.S. Pat. No. 4,861,800); Alzheimer's disease and Parkinson's disease, particularly through the use of transdermal dosage forms, including ointments, creams and patches; macular degeneration (U.S. Pat. No. 5,242,950); age-dependent degeneracies, including renal function and cognitive function as evidenced by spatial learning ability (U.S. Pat. No. 5,151,449); pituitary-dependent Cushing's disease in humans and nonhumans (U.S. Pat. No. 5,192,808); immune system dysfunction in both humans (U.S. Pat. No. 5,387,615) and animals (U.S. Pat. No. 5,276,057); age-dependent weight loss in mammals (U.S. Pat. No. 5,225,446); schizophrenia (U.S. Pat. No. 5,151,419); and various neoplastic conditions including cancers, such as mammary and pituitary cancers. WO 92/17169 discloses the use of selegiline in the treatment of neuromuscular and neurodegenerative disease and in the treatment of CNS injury due to hypoxia, hypoglycemia, ischemic stroke or trauma. In addition, the biochemical effects of selegiline on neuronal cells have been extensively studied (e.g., see Tatton et al., 1991 and 1993). U.S. Pat. No. 6,562,365 discloses the use of desmethylselegiline for selegiline-responsive diseases and conditions.

Selegiline (1-deprenyl) is a selective MAO-B inhibitor which is a useful anti-Parkinson drug both in monotherapy and as an adjunct to L-DOPA therapy, and has L-DOPA sparing action (Birkmayer et al., 1977; Riederer and Rinne, 1992).

Selegiline is a propargyl derivative of 1-methamphetamine and thus its major metabolite is 1-methamphetamine (Szoko et al., 1999; Kraemer and Maurer, 2002; Shin, 1997), which is neurotoxic (Abu-Raya et al., 2002; Am et al., 2004). In contrast to aminoindan, a rasagiline metabolite, L-methamphetamine prevents the neuroprotective activities of rasagiline and selegiline in partially differentiated cultured PC-12 cells (Am et al., 2004).

Selegiline and methamphetamine unlike rasagiline and aminoindan, have sympathomimetic activity (Simpson, 1978) that increases heart rate and blood pressure (Finberg et al., 1990; Finberg et al., 1999). Recent studies (Glezer and Finberg, 2003) have indicated that the sympathomimetic action of selegiline can be attributed to its 1-methamphetamine and amphetamine metabolites. These properties are absent in rasagiline and in its metabolite aminoindan. Parkinsonian patients receiving combined treatments with selegiline plus levodopa have been reported to have a higher mortality rate than those treated with levodopa alone (Lees, 1995). This is not related to the MAO-B inhibitory activity of selegiline, but is rather attributed to its sympathomimetic action and methamphetamine metabolites (Reynolds et al., 1978; Lavian et al., 1993).

Several propargylamine derivatives have been shown to selectively inhibit MAO-B and/or MAO-A activity and, thus to be suitable for treatment of neurodegenerative diseases such as Parkinson's and Alzheimer's disease. In addition, these compounds have been further shown to protect against neurodegeneration by preventing apoptosis.

U.S. Pat. No. 5,169,868, U.S. Pat. No. 5,840,979 and U.S. Pat. No. 6,251,950 disclose aliphatic propargylamines as selective MAO-B inhibitors, neuroprotective and cellular rescue agents. The lead compound, (R)—N-(2-heptyl)methyl-propargylamine (R-2HMP) has been shown to be a potent MAO-B inhibitor and antiapoptotic agent (Durden et al., 2000).

Propargylamine was reported many years ago to be a mechanism-based inhibitor of the copper-containing bovine plasma amine oxidase (BPAO), though the potency was modest. U.S. Pat. No. 6,395,780 discloses propargylamine as a weak glycine-cleavage system inhibitor.

As demonstrated by previous publications of the present inventors, the neuroprotective and anti-apoptotic efficacies of rasagiline are similar to those of its S-enantiomer, the non-monoamine inhibitor TVP1022, suggesting that neuroprotection is not due to MO inhibition (Youdim and Weinstock, 2001; Youdim et al, 2003). In fact, since N-propargylamine itself has a similar mode of action with the same potency as that of rasagiline and TVP1022, the neuroprotective effects were assigned to the propargyl moiety of these drugs (Youdim and Weinstock, 2001; Weinreb et al., 2004). Hence, rasagiline and related propargylamines suppress the apoptotic neuronal death cascade initiated by the mitochondria, and prevent the pro-apoptotic decline in mitochondrial membrane potential (ΔΨm) due to permeability transition. Furthermore, these drugs inhibit the activation of apoptotic processes including activation of caspase 3, nuclear translocation of glyceraldehyde-3-phosphate dehydrogenase, and nucleosomal DNA fragmentation (Youdim and Weinstock, 2001; Youdim et al., 2003), and increase the expression of the anti-apoptotic Bcl-2 and Bcl-xL proteins (Weinreb et al., 2004; Akao et al., 2002).

Copending U.S. patent application Ser. No. 10/952,379, entitled “Use of propargylamine as neuroprotective agent”, filed on Sep. 29, 2004 (US 20050191348), discloses that propargylamine exhibits neuroprotective and anti-apoptotic activities and can, therefore, be used for all known uses of rasagiline and similar drugs containing the propargylamine moiety.

Copending U.S. patent application Ser. No. 11/244,150, entitled “Methods for treatment of renal failure”, filed on Oct. 6, 2005 (US 20070082958), discloses a method for treatment of a renal failure, either acute or chronic, which comprises administering to the subject an amount of an active agent selected from the group consisting of propargylamine, a propargyl amine derivative, and a pharmaceutically acceptable salt thereof.

All and each of the above-mentioned US patents and patent applications are herewith incorporated by reference in their entirety as if fully disclosed herein.

### SUMMARY OF THE INVENTION

It has been found, in accordance with the present invention, that both propargylamine and its derivative S(−)-N-propargyl-1-aminoindan (TVP1022) markedly attenuated doxorubicin-induced cardiotoxicity in neonatal rat ventricular myocytes (NRVM), as indicated by both inhibiting doxorubicin-induced apoptosis and preventing doxorubicin-induced deleterious effects on ventricular muscle contraction, and importantly, did not interfere with the anti-tumor activity of doxorubicin. Furthermore, TVP1022 was found to increase survival of doxorubicin-treated mice and prevented doxorubicin-induced decrease in both body and heart weights, indicating that these agents can be co-administered with anthracycline chemotherapeutic agents, particularly doxorubicin, in the treatment of different human malignancies, and thus considered as cardioprotective agents against anthracycline-induced cardiotoxicity.

The present invention thus relates to a method for preventing or attenuating anthracycline-induced cardiotoxicity in a patient treated with an anthracycline chemotherapeutic agent, comprising administering to said subject an amount of an active agent selected from the group consisting of propargylamine, a propargylamine derivative, and a pharmaceutically acceptable salt thereof, effective to treat the subject.

The anthracycline chemotherapeutic agent may be any chemotherapeutic agent of the anthracycline family including daunorubicin, doxorubicin, epirubicin, idarubicin and mitoxantrone. In a preferred embodiment, the anthracycline chemotherapeutic agent is doxorubicin.

In one preferred embodiment of the invention, the agent is propargylamine or a pharmaceutically acceptable salt thereof. In another preferred embodiment, the agent is a propargylamine derivative such as an N-propargyl-1-aminoindan, e.g. R(+)-propargyl-1-aminoindan (rasagiline) or its enantiomer S(−)-N-propargyl-1-aminoindan (TVP1022), and analog thereof, or a pharmaceutically acceptable salt thereof.

### BRIEF DESCRIPTION OF THE FIGURES

FIGS. 1A-1B depict apoptosis induced in H9c2 rat heart cells by means of recombinant Fas ligand (rFasL). The apoptotic cells detected by DAPI staining are marked by the arrows (1B). FIG. 1A—control.

FIGS. 2A-2C show that rasagiline, S(−)-N-propargyl-1-aminoindan (TVP1022) and propargylamine block Fas-mediated apoptosis in H9c2 cells. Maximal apoptotic effect of Fas activation, attained at 10 hours incubation with rFasL, was completely prevented by 10 μM rasagiline (2A). The apoptotic effect of Fas activation, attained at ˜10 hours incubation with rFasL, was completely prevented by both TVP1022 (0.1 or 1.0 μM) (2B) and propargylamine (0.1 or 1.0 μM) (2C).

FIGS. 3A-3E show that rasagiline, propargylamine and S(−)-N-propargyl-1-aminoindan (TVP1022) protect against serum starvation-induced apoptosis in H9c2 cells: (3A) maximal apoptotic effect, induced by 9 hours serum starvation, was completely prevented by 10 μM rasagiline; (3B-3D) anti-apoptotic effects obtained by either 0.1-10 μM rasagiline, 0.01-1 μM propargylamine or 0.01-1 μM TVP1022, respectively; (3E) anti-apoptotic effect obtained by 0.1-10 μM TVP1022, using the MTT staining assay as a measure for apoptosis.

FIG. 4 shows that rasagiline protects against serum starvation-mediated but not H₂O₂— induced apoptosis in H9c2 cells (n=4 experiments, ˜2000 cells counted). * compared to control. ** compared to serum starvation (p<0.05).

FIG. 5 shows that both propargylamine and S(−)-N-propargyl-1-aminoindan (TVP1022) block Fas-mediated hypertrophy in cultured neonatal rat ventricular myocytes. The top panel depicts representative atrial natriuretic peptide (ANP) mRNA blots in control, rFasL, rFasL+propargylamine, and in rFasL+TVP1022. The lower panel depicts the summary of three experiments performed with each one of these drugs. Hypertrophy was expressed as the ratio between ANP and actin. *P<0.05 vs. control.

FIGS. 6A-6C show the effect of serum starvation (SS) in cultures of neonatal rat ventricular myocytes (NKVM) on apoptosis induction, indicated by the level of caspase-3 cleavage, and the effect of propargylamine (PA) thereon. (6A) serum starvation causes apoptosis, represented by a marked increase in caspase-3 cleavage. (6B) 0.1 μM propargylamine attenuates serum starvation-induced apoptosis as indicated by decreased level of caspase-3 cleavage (n=3, P<0.01 compared to SS). (6C) 0.1 μM propargylamine attenuates serum starvation-induced apoptosis as indicated by increased expression of Bcl-2 (n=3, P<0.05 compared to SS).

FIGS. 7A-7D show the effect of intravenous administration of S(−)-N-propargyl-1-aminoindan (TVP1022) (either 1 or 10 mg/kg) on the cardiac function in rats: (7A) cardiac output (ml/min); (7B) cardiac index (ml/min*100 gr body weight); (7C) heart rate (beats/min); and (7D) mean arterial pressure (mm/Hg). Recovery=after washout period.

FIGS. 8A-8E show the effects of propargylamine and S(−)-N-propargyl-1-aminoindan (TVP1022) (5 mg/kg/day), orally administered for 21 days, on the expression of mitochondrial Bax, a pro-apoptotic protein, and of mitochondrial Bcl-2 and PKC-ε, both anti-apoptotic proteins. Propargylamine does not affect Bax expression (8A) but increases Bcl-2 expression (8B), resulting in marked increase in the ratio Bcl-2/Bax expression (8C). Propargylamine increases PKC-E expression (8D). TVP1022 increases PKC-ε expression (8E).

FIGS. 9A-9B show that both caspase-3 (9A) and cytochrome C (9B) markedly increase following induction of volume overload, indicating that volume overload-induced CHF is associated with increased expression of these two proteins. Sham-operated rats served as controls.

FIGS. 10A-10B show that both S(−)-N-propargyl-1-aminoindan (TVP1022) and propargylamine significantly reduce CHF-induced increase in caspase-3 and cytosolic cytochrome C, both pro-apoptotic proteins. (10A) Effect of TVP1022 (7.5 mg/kg/day, orally administered for 21 days) on caspase-3 expression in CHF-induced rats (vehicle=untreated CHF rats). (10B) Effect of TVP1022 (1 mg/kg/day) and propargylamine (5 mg/kg/day), orally administered for 21 days, on cytochrome C expression in CHF-induced rats (vehicle=untreated CHF rats).

FIGS. 11A-11C show that S(−)-N-propargyl-1-aminoindan (TVP1022) completely prevents the hypertrophic increase in the diastolic area seen in CHF rats at days 10 and 21 of the treatment protocol, as described in Material and Methods hereinafter.

FIGS. 12A-12C show that S(−)-N-propargyl-1-aminoindan (TVP1022) completely prevents the hypertrophic increase in the systolic area seen in CHF rats at days 10 and 21 of the treatment protocol, as described in Material and Methods hereinafter.

FIGS. 13A-13C show that the fractional shortening in the CHF rats, 14 days post surgical creation of an aorto-caval fistula (AVF), is significantly reduced, but completely prevented by administration of S(−)-N-propargyl-1-aminoindan (TVP1022), as described in Material and Methods hereinafter.

FIGS. 14A-14C show that the administration of propargylamine as described in Material and Methods hereinafter completely prevents the hypertrophic increase in the diastolic (14A) and systolic (14B) areas seen in the CHF rats, 14 days post surgical creation of aortocaval fistula (AVF), as well as a significant reduction in the fractional shortening.

FIGS. 15A-15B show that S(−)-N-propargyl-1-aminoindan (TVP1022) and propargylamine inhibit doxorubicin (Dox)-induced apoptosis and the increase in cleaved caspase 3 levels in NRVM. Cultures were pretreated with TVP1022 (1 μM) or propargylamine (1 μM) for 24 hrs before exposure to doxorubicin (0.5 μM) for additional 24 hrs. Apoptotic nuclei were determined by DAPI staining assay and expressed as fold of control (untreated cultures) (15A). Cleaved caspase-3 was determined by means of immunoblotting analysis in cell lysates and expressed as fold of control (15B). Loading of the lanes was normalized to β-actin levels. Data are expressed as mean ±SEM (n=3). #P<0.001 vs. control; *P<0.05, **P<0.01 vs. doxorubicin.

FIG. 16A-16C shows that S(−)-N-propargyl-1-aminoindan (TVP1022) and propargylamine prevent doxorubicin (Dox)-induced decrease in Bcl-2 protein expression in NRVM. Cultures were treated as described in Example 10 hereinafter. Representative Western blots and quantitative results of Bcl-2 and Bax are shown in 16A and 16B, respectively, while the ratio of Bcl-2/Bax is shown in 16C. Loading of the lanes was normalized to β-actin levels, and the results are presented relative to control levels. Data are expressed as mean ±SEM (n=3). #P<0.001 vs. control; *P<0.05; **P<0.01 vs. doxorubicin.

FIGS. 17A-17E show that S(−)-N-propargyl-1-aminoindan (TVP1022) and propargylamine prevent doxorubicin (Dox)-induced alterations in the [Ca²⁺]ᵢ transients parameters in NRVM. Cultures were pre-incubated with TVP1022 (1 μM) or propargylamine (1 μM) for 24 hrs before adding doxorubicin (0.5 μM) for additional 24 hrs. 17A shows a scheme illustrating the measured [Ca²⁺]ᵢ transients parameters; 17B shows representative [Ca²⁺]ᵢ transients recorded from a control culture and from cultures treated either with doxorubicin alone or with both doxorubicin and S(−)-N-propargyl-1-aminoindan; 17C shows diastolic [Ca²⁺]ᵢ expressed as Fura-2 fluorescence ratio; 17D shows the time constant (τ, sec) of the [Ca²⁺]ᵢ transient relaxation calculated from the equation y=y₀+A₁e^{-t/τ}; and 17E shows the [Ca²⁺]ᵢ transient amplitude (systolic ratio−diastolic ratio, in arbitrary units). In each group, n=5 cultures. #P<0.01 vs. control; *P<0.05, **P<0.01 vs. doxorubicin.

FIG. 18A-18D show that S(−)-N-propargyl-1-aminoindan (TVP1022) and propargylamine prevent doxorubicin (Dox)-induced alterations in the contraction parameters in NRVM. Cultures were treated as described in Example 11 hereinafter. 18A shows a scheme illustrating the measured contraction parameters; 18B shows representative contractions recorded from a control culture and from cultures treated with either with doxorubicin alone or with both doxorubicin and S(−)-N-propargyl-1-aminoindan; 18C shows the maximal rate of myocyte contraction; and 18D shows the maximal rate of myocyte relaxation. In each group, n=5 cultures. #P<0.05 vs. control; *p<0.05 vs. Doxorubicin.

FIGS. 19A-19B show that S(−)-N-propargyl-1-aminoindan (TVP1022) does not cause cancer cells proliferation in human cervical carcinoma HeLa (19A) and breast carcinoma MDA-231 (19B) cells. Cells were incubated with or without TVP1022 (0.01, 0.1 or 1 μM), for 49 hrs, and cells proliferation was determined by the MTT staining assay (n=4 experiments, each performed in triplicates).

FIGS. 20A-20B show that S(−)-N-propargyl-1-aminoindan (TVP1022) does not interfere with the anti-cancer activity of doxorubicin (Dox) in human cervical carcinoma HeLa (20A) and breast carcinoma MDA-231 (20B) cells. Cells were pre-incubated with or without TVP1022 (0.01, 0.1 or 1 μM) for 24 hrs, and then treated with doxorubicin (1 μM in the case of HeLa cells and 10 μM in the case of MDA-231 cells) for additional 24 hrs, and cell viability was determined by the MTT staining assay. *p<0.05 vs. Control (n=4 experiments, each performed in triplicates).

FIGS. 21A-21C show that both S(−)-N-propargyl-1-aminoindan (TVP1022) and propargylamine do not interfere with the anti-cancer activity of doxorubicin (Dox) in human cervical carcinoma HeLa (21A), breast carcinoma MDA-231 (21B) and breast cancer MDA-415 (21C) cells. Cells were pre-incubated with or without TVP1022 (1 μM) or propargylamine (1 μM) and then treated with doxorubicin (10 μM) for additional 24 hrs. Cell viability was determined by the MTT staining assay and expressed as percent of untreated control. Data are expressed as mean ±SEM (n=5-6). #P<0.01 vs. control.

FIG. 22 shows that S(−)-N-propargyl-1-aminoindan increases survival of doxorubicin-treated mice. Mice were divided into 5 experimental groups, wherein mice in the doxorubicin group were TV injected with one dose of doxorubicin, 20 mg/kg, into the tail vein (n=22); mice in the control group were untreated (n=9); mice in the sham group were fed with DDW and injected with doxorubicin vehicle (saline) (n=17); mice in the TVP1022 group were fed with TVP1022, 7.5 mg/kg/day, for 15 days (n=13); and mice in the TVP1022+doxorubicin group were fed with TVP1022, 7.5 mg/kg/day, for 15 days, and on day 7 were TV injected with doxorubicin, 20 mg/kg, into the tail vein (n=13).

FIG. 23 shows the average final body weight of the surviving animals in each one of the mice groups mentioned in FIG. 22, namely, the doxorubicin (Dox)-treated group (n=15), the control group (n=11), the sham group (n=9), the TVP1022-treated group (n=13) and the TVP1022+doxorubicin-treated group (n=9). The results are expressed as Mean ±SD. ANOVA analysis showed significant difference between the groups (P<0.01). Using post hoc Bonferroni multiple comparisons showed the following: *P<0.05 doxorubicin vs. sham, control, TVP1022 and TVP1022+doxorubicin groups. **P<0.05 doxorubicin vs. TVP1022, and TVP1022+doxorubicin groups.

FIG. 24 shows the average heart weight of the surviving animals in each one of the mice groups mentioned in FIG. 22, namely, the doxorubicin (Dox)-treated group (n=12), the control group (n=9), the sham group (n=5), the TVP1022-treated group (n=10) and the TVP1022+doxorubicin-treated group (n=7). The results are expressed as Mean ±SD. ANOVA analysis showed significant difference between the groups (P<0.01). Using post hoc Bonferroni multiple comparisons showed the following: *P<0.05 doxorubicin vs. sham, control. TVP1022 and TVP1022+doxorubicin groups. **P<0.05 doxorubicin vs. TVP1022, and TVP1022+doxorubicin groups.

### DETAILED DESCRIPTION OF THE INVENTION

As described in detail in Examples 1-9 hereinafter, both propargylamine and S(−)-N-propargyl-1-aminoindan (also designated TVP1022), which do not inhibit monoamine oxidase, decrease the expression of key pro-apoptotic proteins such as caspase-3 and cytosolic cytochrome C, and increase the expression of anti-apoptotic proteins such as mitochondrial Bcl-2 and PKC-ε, thus shifting the balance between the anti-apoptotic and the pro-apoptotic proteins towards the former and generating anti-apoptotic effect. These studies have been conducted both in in vitro and in vivo experiments, in which both naive and volume overload-induced congestive heart failure (CHF) rats have been used. Furthermore, pretreatment with propargylamine or TVP1022 blocks the volume overload induced hypertrophy in CHF rats and the reduction in ventricular mechanical function as derived from echocardiological parameters.

As further described in Examples 10-13, both propargylamine and its derivative TVP1022 significantly attenuate doxorubicin-induced cardiotoxicity in neonatal rat ventricular myocytes (NRVM) and, importantly, do not interfere with the anti-tumor activity of this anthracycline.

In particular, as shown hereinafter and further supported by previous studies (Jeremias et al., 2005; Ueno et al., 2006; Wu et al., 2002; Green and Leeuwenburgh, 2002; Sawyer et al., 1999; Kotamraju et al., 2000; Spallarossa et al., 2004), doxorubicin causes prominent apoptosis, expressed as nuclear fragmentation, a marked increase in cleaved caspase 3 and a reduction in Bcl-2 protein expression. Recent studies implicated mitochondrial dysfunction as an early event in doxorubicin-induced cardiotoxicity and demonstrated that doxorubicin increases cytochrome C release (Green and Leeuwenburgh, 2002). It is well established that once cytochrome C is released to the cytosol, it binds to apoptotic protease-activating factor 1 (Apafl) and to pro-caspase 9, leading to generation of activated caspase 9, which then activates executioner caspases, mainly caspase 3 that leads to apoptosis (Clerk et al., 2003). Example 10 particularly shows that doxorubicin markedly decreases Bcl-2 protein expression without changing Bax, thus decreases Bcl-2/Bax ratio, which predisposes the cell to apoptotic stimuli. These data confirm previous results showing a decrease in Bcl-2 protein expression following doxorubicin treatment (Wu et al., 2002; Maruyama et al., 2001). Indeed, apoptotic-like cell death is known to play a role in cardiomyopathy induced by doxorubicin (Sawyer et al., 1999; Kalyanaraman et al., 2002; Shizukuda et al., 2005), indicating that inhibitors of apoptosis may provide hope for the prevention/treatment of doxorubicin-induced cardiomyopathy.

Previous studies have shown that propargylamine derivatives such as rasagiline and TVP1022 exhibit a broad cytoprotective activity against a variety of neurotoxins in neuronal cell cultures and in in vivo models. Moreover, propargylamine exerts neuroprotective activity against N-methyl-R-salsolinol and serum deprivation-induced cell death, suggesting its essentiality for neuroprotection (Weinreb et al., 2004; Maruyama et al. 2000).

In view of the notion that the mechanisms of apoptotic cell death of neurons and cardiomyocytes are similar (Mattson and Kroemer, 2003; Pollack et al., 2002), the cardioprotective efficacy of both TVP1022 and propargylamine against doxorubicin-induced apoptosis was studied, and as shown hereinafter, both agents attenuate doxorubicin-induced apoptosis in NRVM, wherein the inhibition of cellular apoptosis is correlated with its inhibitory effects on doxorubicin-induced caspase 3 activation. In addition, both TVP1022 and propargylamine almost completely prevent doxorubicin-induced reduction in the expression of anti-apoptotic Bcl-2 protein, thus increase Bcl-2/Bax ratio and eventually protect myocytes from a mitochondria-mediated apoptosis.

These observations are consistent with recent studies of the present inventors, demonstrating that activation/regulation of PKC in association with Bcl-2 protein family promotes neuronal survival by rasagiline and by its propargyl moiety (Weinreb et al., 2004). As found in those studies, rasagiline suppresses cell death by preventing the activation of the mitochondrial apoptotic cascade in response to the neurotoxins SIN-1 and N-methyl-R-salsolinol (Akao et al., 2002; Yi et al., 2006), and its neuroprotective efficacy does not depend on inhibition of MAO-B, but is rather associated with some intrinsic pharmacological action of the propargyl moiety acting on mitochondria cell survival proteins (Yi et al., 2006; Youdim et al., 2001b; Youdim et al., 2005).

As shown in Example 11, and supported by previous reports (Maeda et al., 1999; Mijares and Lopez, 2001; Shneyvays et al., 2001; Wang et al., 2001; Fixler et al., 2002; Timolati et al., 2006), doxorubicin adversely affects [Ca²⁺]ᵢ transients and contractions. In particular, it elevates diastolic [Ca²⁺]ᵢ and slows the kinetics of [Ca²⁺]ᵢ transient relaxation, and concomitantly, decreases the maximal rates of contraction and relaxation. As suggested, the deleterious effects of doxorubicin on the contraction are mediated by changes in ion currents composing the transmembrane action potential, [Ca²⁺]ᵢ handling and contractile proteins. The proposed mechanisms underlying the toxic effects of doxorubicin, which can account for its deleterious effects described herein, are exemplified by the following findings: (i) Doxorubicinol, a major metabolite of doxorubicin, impaired cardiac contractility in guinea pig ventricular myocytes by both shortening action potential duration due to activation of Iₖ and by partially depleting sarcoplasmic reticulum Ca²⁺ content, leading to reduced amounts of Ca²⁺ available for contraction (Wang et al., 2001); (ii) In cultured NRVM (Friberg and Wieloch, 2002), adult rat ventricular cardiomyocytes (Timolati et al., 2006) and rabbit in vivo model (Arai et al., 1998; Olson et al. 2005), doxorubicin decreased the expression of the sarcoplasmic reticulum Ca²⁺ transporting ATPase (SERCA2) at the mRNA and protein levels; (iii) Doxorubicin caused partial degradation and decreased SERCA2 function in NRVM (Arai et al., 2000) and in adult rat ventricular cardiomyocytes (Timolati et al., 2006), as well as in animal models (Arai et al., 1998; Olson et al., 2005); (iv) Doxorubicin decreased mRNA and protein expression of the ryanodine receptor 2 (RyR2) in the rabbit in vivo model (Arai et al., 1998; Olson et al., 2005). Furthermore, doxorubicin reduced [³H]-ryanodine binding (Halestrap et al., 2004; Pollack et al., 2002) and increased RyR open probability (Feng et al., 1999), which may lead to reduced sarcoplasmic reticulum Ca²⁻ content and increased diastolic [Ca²⁺]ᵢ, as was demonstrated in the present study; and (v) Doxorubicin decreased the protein expression of the cardiac Na/Ca exchanger (NCX), phospholamban and calcequestrin in the rabbit model (Arai et al., 1998; Olson et al., 2005).

As shown hereinafter, further to their ability to attenuate doxorubicin-induced apoptosis, both TVP1022 and propargylamine, at 1 μM, completely prevent the various deleterious effects of doxorubicin on the [Ca²⁺]ᵢ transients and contractions, suggesting that they may prevent the systolic and diastolic dysfunction in patients treated with anthracyclines.

As shown in Example 13, exemplified using three human cancer cell lines: HeLa, MDA-231 and MDA-415, both TVP1022 and propargylamine do not interfere with the marked anti-cancer efficacy of doxorubicin. Example 14 further shows that TVP1022 increases survival of doxorubicin-treated mice and prevents doxo-induced decrease in both body and heart weights.

The aforesaid findings indicate that both agents can be safely co-administered with anthracycline chemotherapeutic agents, particularly doxorubicin, in the treatment of different human malignancies, without the concern of diminished doxorubicin therapeutic efficacy, and thus may be considered as cardioprotective agents against anthracycline-induced cardiotoxicity.

The present invention thus relates to a method for preventing or attenuating anthracycline-induced cardiotoxicity in a patient treated with an anthracycline chemotherapeutic agent, comprising administering to said subject an amount of an active agent selected from the group consisting of propargylamine, a propargylamine derivative, and a pharmaceutically acceptable salt thereof, effective to treat the subject.

In one embodiment, the method of the present invention is for preventing anthracycline-induced cardiotoxicity in a patient treated with an anthracycline chemotherapeutic agent.

In another embodiment, the method of the present invention is for attenuating anthracycline-induced cardiotoxicity in a patient treated with an anthracycline chemotherapeutic agent.

The method of the invention is suitable for preventing and/or attenuating both acute and chronic anthracycline-induced cardiotoxicity.

In one preferred embodiment, the active agent used in the present invention is propargylamine or a pharmaceutically acceptable salt thereof. The use of any physiologically acceptable salt of propargylamine is encompassed by the present invention such as the hydrochloride, hydrobromide, sulfate, mesylate, esylate, tosylate, sulfonate, phosphate, or carboxylate salt. In more preferred embodiments, propargylamine hydrochloride and propargylamine mesylate are used according to the invention.

In another preferred embodiment, the active agent used in the present invention is N-propargyl-1-aminoindan, either in its racemic form (described, for example, in U.S. Pat. No. 6,630,514) or as the R-enantiomer R(+)-N-propargyl-1-aminoindan (rasagiline, described, for example, in U.S. Pat. No. 5,387,612) or as the S-enantiomer S-(−)-N-propargyl-1-aminoindan (TVP1022, described, for example, in U.S. Pat. No. 6,277,886). In a more preferred embodiment of the invention, the active agent is rasagiline, the R(+)-N-propargyl-1-aminoindan, or its enantiomer S(−)-N-propargyl-1-aminoindan.

In another preferred embodiment, the active agent is a pharmaceutically acceptable salt of N-propargyl-1-aminoindan or of an enantiomer thereof including, but not limited to, the mesylate, maleate, fumarate, tartrate, hydrochloride, hydrobromide, esylate, p-toluenesulfonate, benzoate, acetate phosphate and sulfate salts. In preferred embodiments, the salt is a pharmaceutically acceptable salt of R(+)-N-propargyl-1-aminoindan such as, but not limited to, the mesylate salt (described, for example, in U.S. Pat. No. 5,532,415), the esylate and the sulfate salts (both described, for example, in U.S. Pat. No. 5,599,991), and the hydrochloride salt (described, for example, in U.S. Pat. No. 6,630,514) of R(+)-N-propargyl-1-aminoindan or S(−) -N-propargyl-1-aminoindan.

In a further embodiment, the active agent is an analog of N-propargyl-1-aminoindan, an enantiomer or a pharmaceutically acceptable salt thereof. In one embodiment, the analogs are the compounds described in U.S. Pat. No. 5,486,541 such as, but not limited to, the compounds 4-fluoro-N-propargyl-1-aminoindan, 5-fluoro-N-propargyl-1-aminoindan, 6-fluoro-N-propargyl-1-aminoindan, an enantiomer thereof and pharmaceutically acceptable addition salts thereof. In another embodiment, the analogs are the compounds described in U.S. Pat. No. 6,251,938 such as, but not limited to, the compounds (rac)-3-(N-methyl,N-propyl-carbamyloxy)-α-methyl-N′-propargyl phenethylamine HCl; (rac)-3-(N,N-dimethyl-carbamyloxy)-α-methyl-N′-methyl, N′-propargyl phenethylamine HCl; (rac)-3-(N-methyl,N-hexyl-carbamyloxy)-α-methyl-N′-methyl, N′-propargyl phenethylamine mesylate; (rac)-3-(N-methyl,N-cyclohexyl-carbamyloxy)-α-methyl-N′-methyl,N′-propargylphenethyl HCl; and (S)-3-(N-methyl, N-hexyl-carbamyloxy)-α-methyl-N′-methyl,N′-propargyl phenethylamine ethane-sulfonate. In a further embodiment, the analogs are the compounds described in U.S. Pat. No. 6,303,650 such as, but not limited to, the compounds (rac) 6-(N-methyl, N-ethyl-carbamyloxy)-N′-propargyl-1-aminoindan HCl; (rac) 6-(N,N-dimethyl, carbamyloxy)-N′-methyl-N′-propargyl-1-amino indan HCl; (rac) 6-(N-methyl, N-ethyl-carbamyloxy-N′-propargyl-1-aminotetralin HCl: (rac) 6-(N,N-dimethyl-thiocarbamyloxy)-1-aminoindan HCl; (rac) 6-(N-propyl-carbamyloxy-N′-propargyl-1-aminoindan HC; (rac) 5-chloro-6-(N-methyl, N-propyl-carbamyloxy)-N′-propargyl-1-α-aminoindan HCl; (S)-6-(N-methyl), N-propyl-carbamyloxy)-N′-propargyl-1-aminoindan HCl; and (R)-6-(N-methyl. N-ethyl-carbamyloxy)-N′-propargyl-1-aminoindan hemi-(L)-tartrate, and 6-(N-methyl, N-ethyl-carbamyloxy)-N′-methyl,N′-propargyl-1-aminoindan described in U.S. Pat. No. 6,462,222.

In a still further embodiment, the active agent is an aliphatic propargylamine described in U.S. Pat. No. 5,169,868. U.S. Pat. No. 5,840,979 and U.S. Pat. No. 6,251,950 such as, but not limited to, the compounds N-(1-heptyl)propargylamine; N-(1-octyl)propargylamine; N-(1-nonyl) propargylamine; N-(1-decyl)propargylamine; N-(1-undecyl)propargylamine: N-(1-dodecyl) propargylamine; R—N-(2-butyl) propargylamine; R—N-(2-pentyl) propargylamine; R—N-(2-hexyl)propargylamine; R—N-(2-heptyl)propargylamine; R—N-(2-octyl) propargylamine; R—N-(2-nonyl)propargylamine; R—N-(2-decyl) propargylamine, R—N-(2-undecyl)propargylamine; R—N-(2-dodecyl)propargylamine: N-(1-butyl)-N-methylpropargylamine; N-(2-butyl)-N-methylpropargylamine; N-(2-pentyl)-N-methylpropargylamine; N-(1-pentyl)-N-methylpropargylamine; N-(2-hexyl)-N-methylpropargylamine; N-(2-heptyl)-N-methylpropargylamine; N-(2-decyl)-N-methylpropargylamine; N-(2-dodecyl)-N-methylpropargylamine; R(−)-N-(2-butyl)-N-methylpropargylamine- or a pharmaceutically acceptable salt thereof.

In yet another embodiment, the active agent is selegiline, desmethylselegiline or norprenyl, pargyline or chlorgyline.

In still another embodiment, the active agent is the compound N-methyl-N-propargyl-10-aminomethyl-dibenzo[b,f]oxepin (known as CGP 3466, described in Zimmermann et al., 1999).

All the US patents and other publications mentioned hereinabove are hereby incorporated by reference in their entirety as if fully disclosed herein.

In another aspect, the present invention provides a pharmaceutical composition for preventing or attenuating anthracycline-induced cardiotoxicity comprising a pharmaceutically acceptable carrier and an agent selected from the group consisting of propargylamine, a propargylamine derivative, and a pharmaceutically acceptable salt thereof as described above.

The pharmaceutical composition provided by the present invention may be in solid, semisolid or liquid form and may further include pharmaceutically acceptable fillers, carriers or diluents, and other inert ingredients and excipients. The composition can be administered by any suitable route, e.g. intravenously, orally, parenterally, rectally, or transdermally. The dosage will depend on the state of the patient and the cardiotoxicity severity, and will be determined as deemed appropriate by the practitioner.

In one embodiment, the pharmaceutically acceptable carrier is a solid and the pharmaceutical composition is in a suitable form for oral administration including tablets, compressed or coated pills, dragees, sachets, hard or soft gelatin capsules, and sublingual tablets. In a preferred embodiment, the pharmaceutical composition is a tablet containing an amount of the active agent in the range of about 0.1-100 mg, preferably from about 1 mg to about 10 mg.

In a more preferred embodiment, the pharmaceutically acceptable carrier is a liquid and the pharmaceutical composition is an injectable solution. The amount of the active agent in the injectable solution is in the range of from about 0.1 mg/kg to about 100 mg/kg, more preferably 1 mg/kg to about 10 mg/kg.

For parenteral administration the invention provides ampoules or vials that include an aqueous or non-aqueous solution or emulsion. For rectal administration there are provided suppositories with hydrophilic or hydrophobic (gel) vehicles.

The methods of the invention are for preventing or attenuating anthracycline-induced cardiotoxicity. In a preferred embodiment, the anthracycline chemotherapeutic agent is doxorubicin.

The dosage and frequency of administration of the drug will depend on the age and condition of the patient, as well as the dosage of the anthracycline chemotherapeutic agent administered and/or the cardiotoxicity severity, and will be determined according to the physician's judgment. It can be presumed that for preventive treatment of patients treated with anthracycline chemotherapeutic agent lower doses will be needed while higher doses will be administered in cases of chronic anthracycline-induced cardiotoxicity. Furthermore, pretreating cancer patients with the active agents of the present invention will enable to use higher doses of doxorubicin for longer periods of time, thus attaining higher anti-cancer efficacy.

The following examples illustrate certain features of the present invention but are not intended to limit the scope of the present invention.

### EXAMPLES

### Materials and Methods

(i) Materials. Propargylamine, as well as rasagiline and its enantiomer S(−)-N-propargyl-1-aminoindan (also designated here TVP1022), were kindly donated by Teva Pharmaceutical Industries Ltd. (Petach Tikva, Israel). Lab-Tek Chamber Slide system and culture plates were purchased from Nalge Nunc International (NY, USA); electrophoresis reagents were purchased from Invitrogen Corporation (Carlsbad, Calif.); cell culture reagents were purchased from Biological Industries, Beth-Haemek (Israel); mounting medium for fluorescence with DAPI were purchased from Vector Laboratories (Inc. Burlingame, Calif., U.S.A); antibodies against caspase 3 and Bax were purchased from Cell Signalling (Beverly, Mass., USA); and Bcl-2 antibody were purchased from BD, Biosciences Transduction Laboratories (Heidelberg, Germany). β-actin antibody and all other reagents were purchased from Sigma Chemical Co. (St. Louis, Mo., USA).

(i>) Cell line H9c2. Experiments were performed on the embryonic rat heart cell line H9c2. H9c2 cells were cultured in Dulbecco's Modified Eagle's Medium (DMEM) (Biological Industries, Beit-Haemek, Israel) supplemented with 10% fetal calf serum (FCS), 50 units/ml penicillin G, 50 μg/ml streptomycin sulfate, 2 mg/ml L-glutamine and sodium pyruvate. H9c2 cells were harvested by trypsinization, washed with phosphate buffered saline (PBS), diluted to a concentration of 5×10⁴ cells/ml with DMEM (high glucose) and cultured at 0.5 ml/well on sterile glass cover slips in 24-well plates.

(iii) NRVM cultures. NRVM cultures were prepared from ventricles of 1-2 day old Sprague-Dawley rats as described by Rubin et al. (1995). Briefly, the ventricles of the excised hearts were dissociated with 0.1% RDB (IIBR, Israel). The dispersed cells were re-suspended in F-10 culture medium containing 1 mM CaCl₂, 100 U/ml penicillin-streptomycin, 5% FCS, 5% donor horse serum, and 25 mg 5-bromo-2-deoxyuridine (BrdU). The cells were preplated for 1 hr to reduce fibroblasts content, and the cell suspension was diluted to a final desired concentration. Cells were seeded in 2-well Permanox Slide (12.5×10⁴ cells/cm²) or in 6-well plates (16×10⁴ cells/cm²) precoated with collagen type I from calf skin (Sigma, C-8919), diluted 1:10 in 0.1 M acetic acid. Thereafter, the cultures were incubated at 37° C. in a humidified atmosphere containing 5% CO₂. At day 4-6 after plating, the regular culture medium was replaced with a culture medium containing 0.5% serum (0.25% FCS, 0.25% donor horse serum), with or without drugs for 24 hrs. Thereafter, doxorubicin was added to a final concentration of 0.5 μM, for 24 hrs.

(iv) Human cancer cell lines. The human cancer cell lines used were cervical carcinoma HeLa, breast carcinoma MDA-231 and breast carcinoma MDA-415. All cell lines were cultured in DMEM supplemented with 10% FCS, 100 U/ml penicillin-streptomycin and 1% L-glutamine, and were incubated at 37° C. in a humidified atmosphere containing 5% CO₂.

(v) Protocols Inducing Apoptosis

(a) H₂O₂ Incubation protocol—To induce apoptosis, H9c2 cultures were exposed to H₂O₂ (0.5 μM) for 7 hours.

(b) Serum starvation—To induce apoptosis, H9c2 cultures were incubated in the culture medium containing 0% FCS for the indicated times.

(c) Activation of the Fas receptor—Fas activation was induced by incubating the cultures with recombinant human Fas Ligand (rFasL; 10 ng/ml) plus the enhancing antibody (1 μg/ml) for the indicated times, according to the manufacturer's recommendations (Alexis Biochemicals, San Diego, Calif.).

(vi) Determination of apoptosis by DAPI. Cultures were counterstained with 4′, 6-diamidino-2-phenylindole (DAPI) to visualize the nuclear morphology. Briefly, cultures were fixed for 20 minutes with 4% paraformaldehyde, permeabilized by 5 minutes incubation with Triton X-100 (0.1% in 0.1% sodium citrate) and washed three times with PBS (pH 7.4). Thereafter, a drop of the mounting solution containing DAPI was added to each slide. The slides were visualized using an Axioscop 2 (Zeiss) upright fluorescence microscope. Cells were scored as apoptotic, only if they exhibited unequivocal nuclear chromatin condensation and fragmentation. The apoptotic rate was expressed as percentage of total counted nuclei.

(vii) Cell viability assay (MTT). The cells were placed in microtiter plates (96 wells) at a density of 25,000 cells/well and allowed to attach for 24 hrs before treatment. Cell viability was measured by means of the 3-(4.5-dimethylthiazol-2-yl)-2.5-diphenyltetrazolium (MTT) test, by adding MTT (5 g/l) to each well for 2 hrs at 37° C. The dissolving buffer containing 20 gr SDS in 100 μl 50% dimethylformamide was brought to pH 4.7 by adding 80% acetic acid and 1 N HCl, and was then added to each well and incubated overnight at 37° C. at humidified atmosphere containing 5% CO₂. The absorbance was detected at 630 nm using Zenyth 2000 Microplate Reader (Harvard Bioscience Company, Austria).

(viii) Western blot analysis. Lysates were prepared from NRVM cultures using RIPA (20 mM Tris-HCl, pH 7.4, 200 mM NaCl, 1% Triton X-100, 0.1% SDS, 0.2% sodium deoxycholate, 5 mM EDTA, 1% Phosphatase inhibitor) containing cocktail protease inhibitor (Roche), and the protein concentration was determined by the Bradford assay. A 20-25 μg sample of total cellular protein was loaded on 12% SDS-PAGE, followed by blotting into polyvinylidene difluoride membranes (Millipore), which were stained by Ponceau concentrate to verify equal loading of protein. Membranes were blocked with 5% dry milk in DDW and 0.05% Tween 20 in TBS for 1 hr. Primary antibodies were diluted in TBS containing 0.05% Tween 20 and incubated with membranes for 24 hrs at 4° C. followed by incubation (1 hr at room temperature) in dilutions of horseradish peroxidase-conjugated secondary antibodies in the same buffer. Following antibody incubations, membranes were washed in 0.5% Tween 20 in TBS. Detection was performed using Western blotting detection reagent, ECL (Amersham, Pharmacia, Little Chalfort Buckinghamshire, UK). Quantification of the results was accomplished by measuring the optical density of the labeled bands from the autoradiograms, using the computerized imaging program Bio-1D (Vilber Lourmat Biotech. Bioprof, France). The values were normalized to β-actin intensity levels.

(ix) Measurement of [Ca²⁺]ᵢ transients and contractions. NRVM cultures were loaded for 30 min at 37° C. with Fura 2-AM (Molecular Probes) at a final concentration of 5 μM in PBS. Excess Fura 2 was removed by rinsing with PBS followed by Tyrode's solution containing (in mM): NaCl 140, KCl 5.4, glucose 10, MgCl₂ 1, sodium pyruvate 2, CaCl₂ 1 and HEPES 10 (pH 7.4 with NaOH). NRVM cultures were then transferred to a nonfluorescent chamber mounted on the stage of an inverted microscope (Diaphot 300, Nikon), and visualized with a x40 oil immersion Neoflour objective. The chamber was perfused with Tyrode's solution at a rate of 1 ml/min. and experiments were performed at 37° C. Fura-2 fluorescence was measured using a dual wavelength system (DeltaScan, Photon Technology International, PTI). Briefly, light emitted from a Xenon arc lamp is fed in parallel into two independent monochromators to obtain quasi-monochromatic light beams of two different wavelengths, exciting the cells at 340 and 380 nm. Either the 340 or 380 nm wavelength was switched by a rotating chopper disk at a frequency enabling ratio measurements at a rate of 100 counts/sec. The two separate monochromators outputs were collected by the ends of a bifurcated quartz fiber optic bundle. The omitted fluorescence (510 nm) was collected by the microscope optics, passed through an interference filter and detected by a photomultiplier tube (710 PMT Photon Counting Detection System, PTI). Using the Felix software (PTI), the raw data were stored for offline analysis, as 340 and 380 counts, and as ratio, R═F₃₄₀/F₃₈₀. To characterize the [Ca²⁺], transient parameters, diastolic and systolic ratios were measured in 10 successive transients and averaged. [Ca²⁺-]ᵢ transient amplitude calculated as systolic ratio minus diastolic ratio. The rate of [Ca²⁺]ᵢ transient relaxation (τ, sec) was calculated from the equation y=y₀+A₁e^{-1/τ} fitted to the relaxation phase. In these experiments, cardiomyocytes were field-stimulated at a frequency of 0.5 Hz using platinum wires embedded in the walls of the perfusion chamber. To monitor myocytes contraction while measuring [Ca²⁺]ᵢ transients, the culture was simultaneously illuminated with red light, and a dichroic mirror (630-nm cutoff) placed in the emission path deflected the cell image to a video optical system (Crescent Electronic). The cursors of the optical system tracked motion of the cell edge along a raster line segment of the image during electrically stimulated contractions. The motion signal obtained at 60 Hz was digitized and stored along with the fluorescence data. To characterize the contraction of NRVM, the maximal rate of contraction, +d(Length)/d(t), and the maximal rate of relaxation, −d(Length)/d(t) were calculated in 10 successive contractions and averaged.

(x) Statistics. Data were expressed as mean ±S.E.M. Data were analyzed by two populations Student's t-test. A level of P<0.05 was accepted as statistically significant.

(xi) Animals.

CHF studies: Studies were conducted on male Sprague Dawley rats (Harlan Laboratories Ltd., Jerusalem, Israel), weighing ˜300 g. The animals were kept in a temperature-controlled room and maintained on standard rat diet (0.5% NaCl). All experiments were performed according to the guidelines of the Technion Committee for Supervision of Animal Experiments (Haifa, Israel). Heart failure was induced by surgical creation of an aortocaval fistula (AVF) between the abdominal aorta and the inferior vena cava (side to side, outer diameter 1-1.2 mm), which is a well established model of volume-overload induced heart failure, featuring many of the clinical symptoms of heart failure and dilated cardiomyopathy in humans. Sham-operated rats served as controls. Drugs (or saline as control) were orally administered, starting 7 days prior to surgery (day 0) and were continued for 21 days. Surgery was performed on day 7 and animals sacrificed 14 days post-surgery (day 21). Cardiac function was determined by echocardiography on days 0, 10 (3 days post-surgery) and 21 (before sacrifice). After the last echocardiography measurement, rats were sacrificed and hearts were analyzed.

Doxorubicin studies: Studies were conducted on male BALB/c mice (Harlan Laboratories Ltd., Jerusalem, Israel) weighing ˜30 g. The animals were kept in a temperature-controlled room and maintained on standard rat diet (0.5% NaCl). All experiments were performed according to the guidelines of the Technion Committee for Supervision of Animal Experiments (Haifa, Israel). Doxorubicin cardiotoxicity was induced by injecting one dose of doxorubicin, 20 mg/kg into the tail vein. Animals were sacrificed 8 days thereafter. Sham-operated mice injected with saline served as controls. TVP1022 (or DDW as control) was orally administered, starting 7 days prior to injecting doxorubicin (day 0) and was continued for 15 days. Hence, doxorubicin was injected on day 7 and animals were sacrificed on day 15.

### Example 1

### Rasagiline, S(−)-N-propargyl-1-aminoindan and propargylamine Protect H9c2 Heart Cells Against Apoptosis Induced by Fas Activation

The first apoptosis-inducing protocol tested was activation of the Fas receptor with recombinant Fas Ligand (rFasL) plus the enhancing antibody (Yaniv et al., 2002).

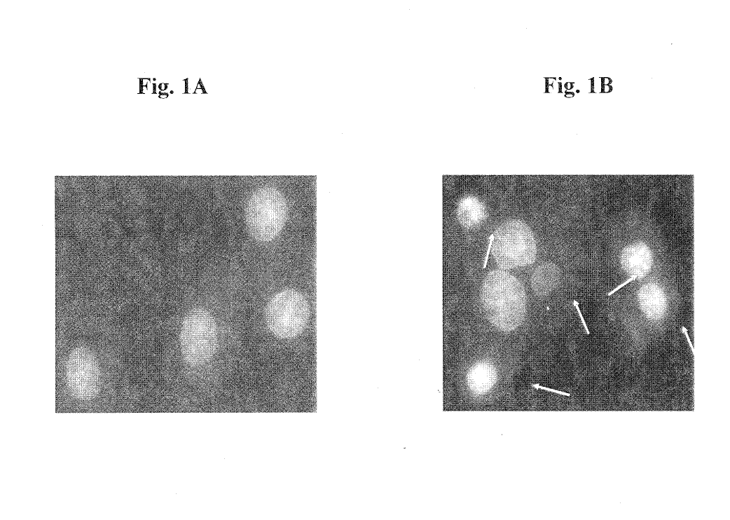

Cultures of embryonic rat heart cell line H9c2 were incubated with rFasL, (10 ng/ml) and an enhancing antibody for periods of time of 9, 10 and 24 hours, and apoptosis measured thereafter. As shown in FIG. 1B, Fas activation caused prominent apoptosis in H9c2 cells, as detected by the DAPI assay.

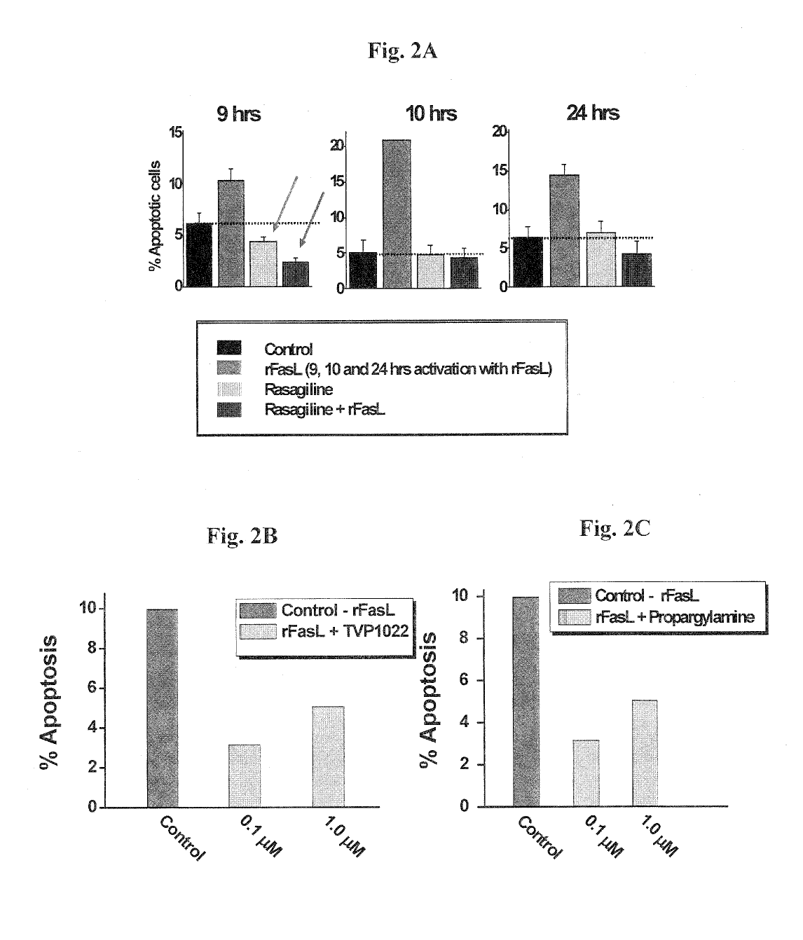

In order to determine whether rasagiline can prevent Fas-mediated apoptosis, the Fas receptor was activated for 9, 10 and 24 hours as described above. Rasagiline (10 μM) was introduced to the culture medium 16 hours before, and was present throughout the apoptosis-inducing protocol (n=3 wells). As seen in FIG. 2A, the maximal apoptotic effect (˜20% apoptosis) of Fas activation was attained at 10 hours incubation with rFasL. This apoptotic effect was completely prevented by rasagiline, demonstrating that rasagiline blocks Fas-mediated apoptosis.

Similar results were obtained using the S-enantiomer, S(−)-N-propargyl-1-aminoindan, and propargylamine. Each one of the drugs, at a concentration of either 0.1 or 1.0 μM was introduced to the culture medium 16 hours before, and was presented throughout the apoptosis-inducing protocol (n=3 wells). As shown in FIGS. 2B-2C, the Fas-mediated apoptosis was ˜10%, attained at ˜10 hours incubation with rFasL, and it was completely prevented by both S(−)-N-propargyl-1-aminoindan (2B) and propargylamine (2C).

### Example 2

### Rasagiline, S(−)-N-propargyl-1-aminoindan and propargylamine Protect H9c2 Heart Cells Against Apoptosis Induced by Serum Starvation

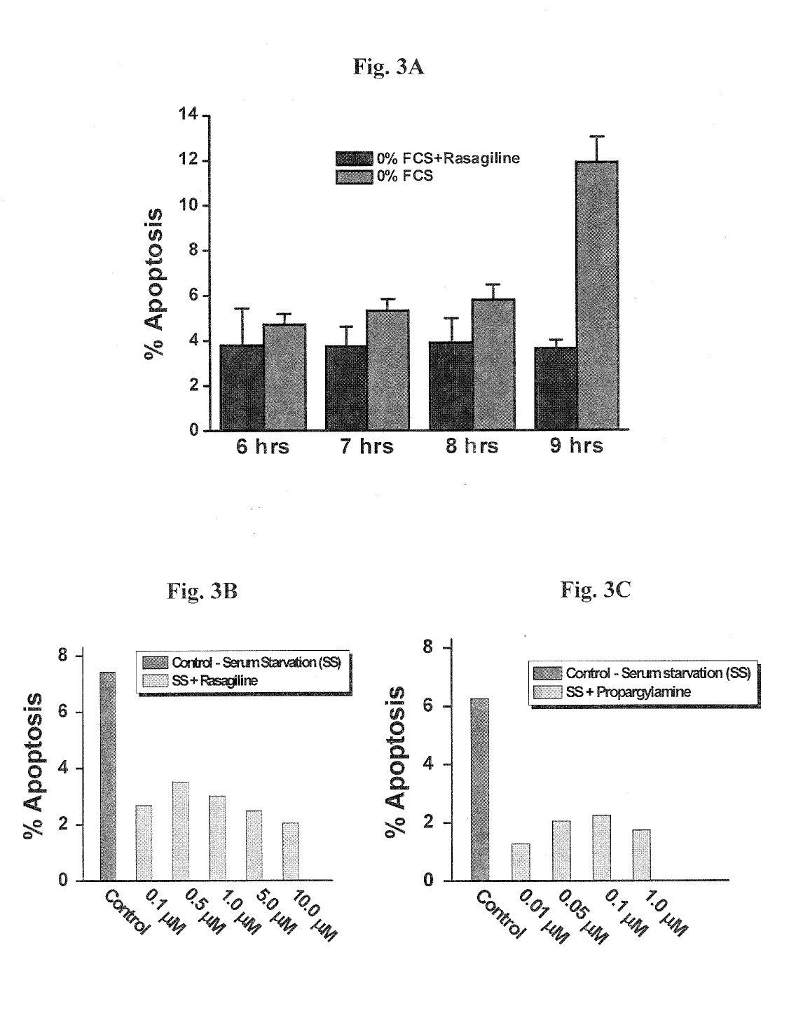

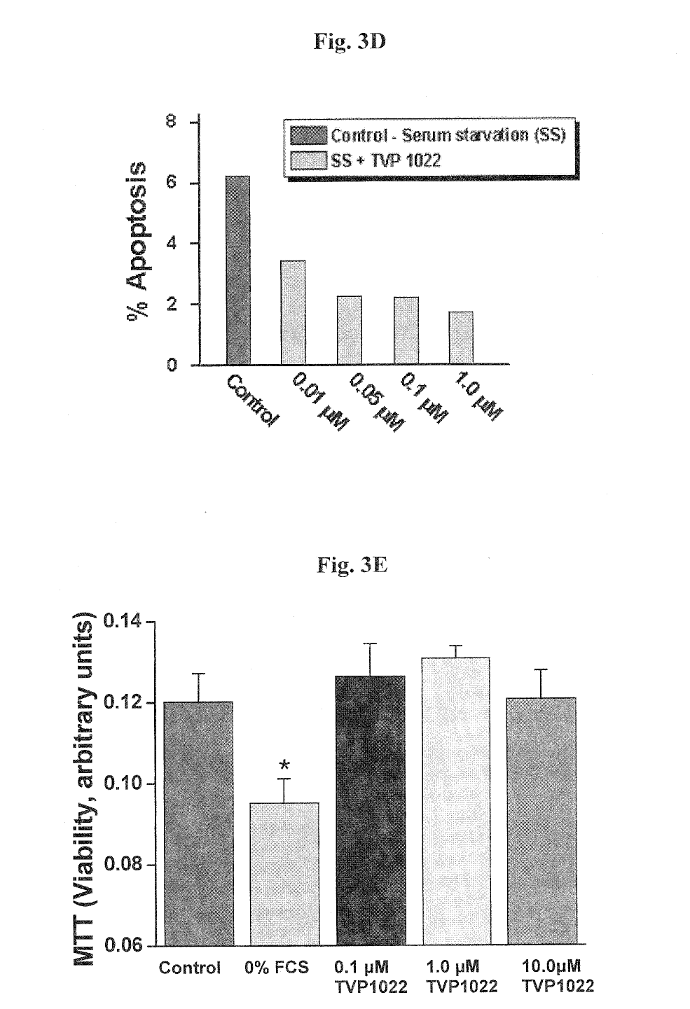

The next apoptosis-inducing stimulus tested was serum starvation (24 hrs, 0% serum in the culture medium). To induce apoptosis, H9c2 cells were incubated in the culture medium containing 0% FCS for 6, 7, 8 or 9 hours. Rasagiline (10 μM) was introduced to the culture medium 2 hours before inducing serum starvation and was present throughout the apoptosis-inducing protocol (n=3 wells). As seen in FIG. 3A, the most effective protocol was 9 hrs serum starvation, which caused 12% apoptosis. This effect was completely prevented by rasagiline.

In the next stage, H9c2 cells were incubated in the culture medium containing 0% FCS for 24 hours, and the anti-apoptotic effect obtained by various concentrations of rasagiline. S(−)-N-propargyl-1-aminoindan and propargylamine was measured. FIG. 3B shows the anti-apoptotic effect obtained by rasagiline (0.1-10 μM) introduced to the culture medium 2 hours before serum starvation. FIGS. 3C-3D show that similar anti-apoptotic effects were obtained by either S(−)-N-propargyl-1-aminoindan or propargylamine (0.01-1 μM), respectively, and FIG. 3E shows the anti-apoptotic effect obtained by S(−)-N-propargyl-1-aminoindan (0.1-10 μM) using the MTT (3-(4,5-dimethylthiazol-2-yl)-2,5-diphenyltetrazolium bromide) staining assay as a measure for apoptosis.

### Example 3

### Rasagiline Protects H9c2 Heart Cells Against Apoptosis Induced by Serum Starvation But not H₂O₂-Induced Apoptosis

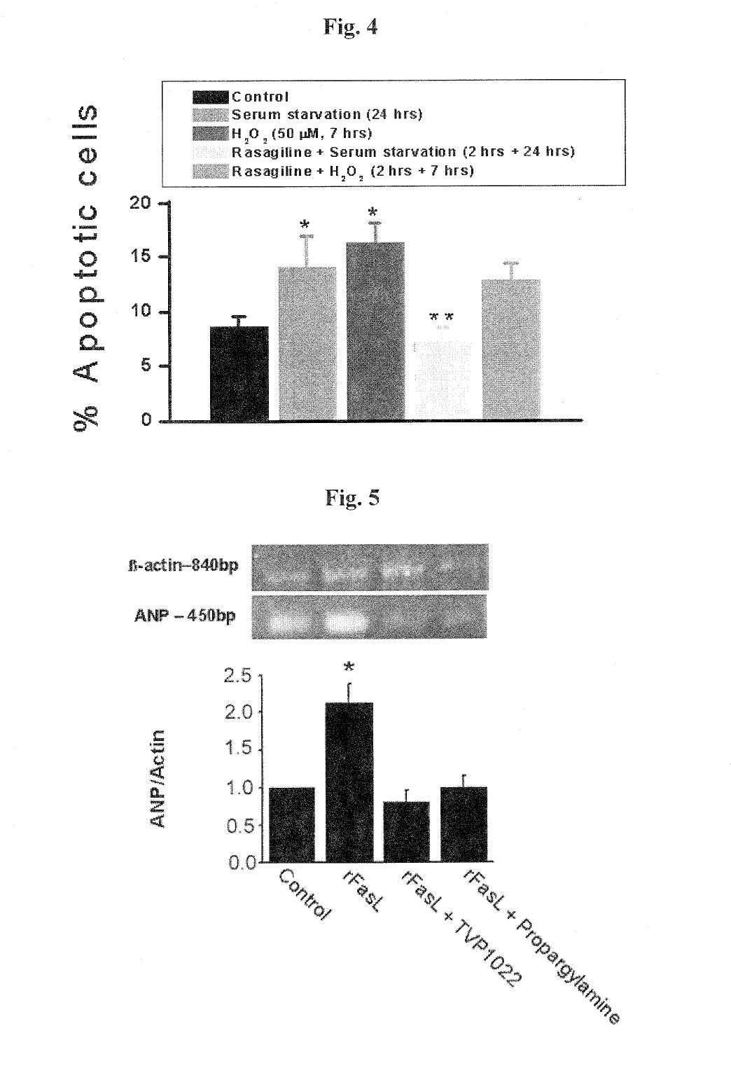

In another experiment, we repeated the serum starvation protocol, and also tested in the same cultures whether rasagiline can protect against H₂O₂-induced apoptosis. Rasagiline was introduced to the culture medium 2 hours before inducing serum starvation or adding H₂O₂, and was present throughout the apoptosis-inducing protocol (n=4 experiments; ˜2000 cells counted). As clearly shown in FIG. 4, rasagiline prevented the apoptosis induced by serum starvation (green bar), but not by H₂O₂ (gray bar).

### Example 4

### Rasagiline, S(−)-N-propargyl-1-aminoindan and propargylamine Block Hypertrophy Induced by Activation of the Fas Receptor in Cultures of Neonatal Rat Ventricular Myocytes

In neonatal rat ventricular myocytes (NKVM), activation of the Fas receptor does not cause apoptosis, but induces marked hypertrophy.

In order to test whether rasagiline can prevent the marked hypertrophy induced in cultured neonatal rat ventricular myocytes (for methods, see Yaniv et al., 2002), Fas was activated for 24 hours by incubation with rFasL, (10 ng/ml plus 1 μg/ml of the enhancer antibody). Hypertrophy was assessed by determining the mRNA levels (by means of RT-PCR) of the atrial natriuretic peptide (ANP), which is a most common molecular marker of hypertrophy. Rasagiline (10 μM/ml) was added to the culture 1 hour before Fas activation and remained in the medium throughout the 24 hours exposure to rFasL. In these preliminary experiments we have found that rasagiline prevented Fas-mediated hypertrophy (data not shown).

In order to test whether S(−)-N-propargyl-1-aminoindan and propargylamine have the same effect on marked hypertrophy induced in cultured neonatal rat ventricular myocytes, similar experiments were performed using either propargylamine or S(−)-N-propargyl-1-aminoindan (both at a concentration of 10 μM) instead of rasagiline. As shown in FIG. 5, the marked ANP mRNA elevation induced by Fas activation for 24 hours was completely blocked by both S(−)-N-propargyl-1-aminoindan and propargylamine (3 experiments per each drug).

Based on these experiments we conclude that rasagiline, S(−)-N-propargyl-1-aminoindan and propargylamine protect ventricular myocytes against hypertrophy caused by activation of the Fas receptor, a finding which may have an important clinical significance.

### Example 5

### Propargylamine Protects Cultured Neonatal Rat Ventricular Myocytes Against Serum Starvation-Induced Apoptosis

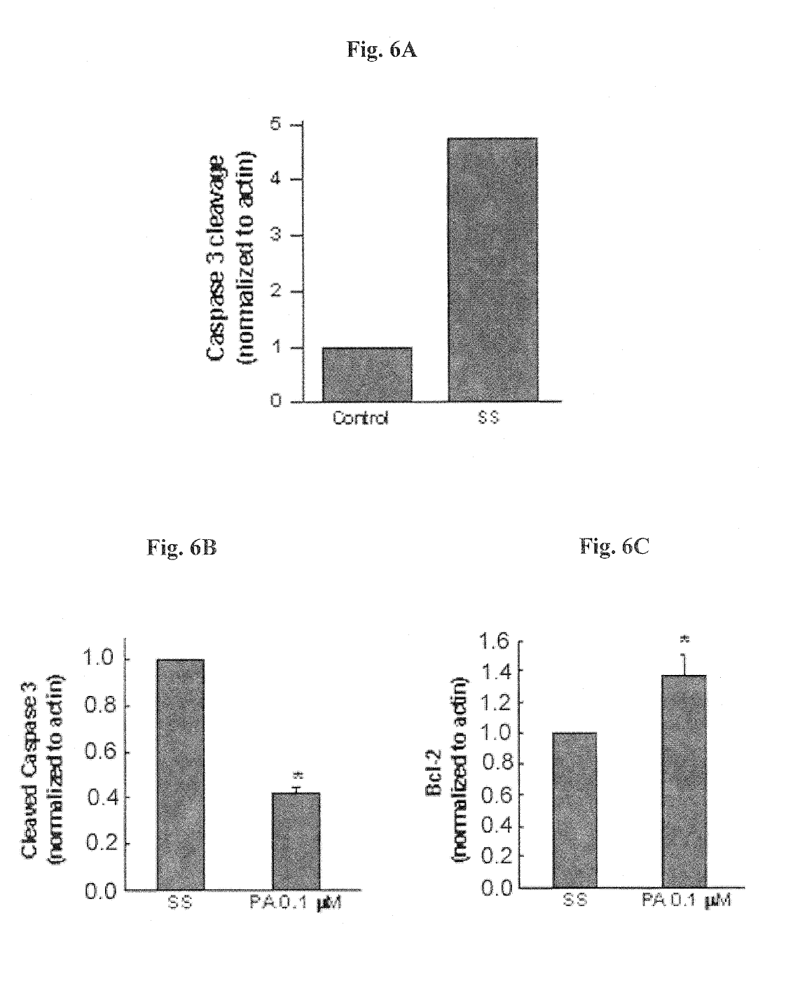

Caspase-3 is a protein of the cysteine-aspartic acid protease (caspase) family, known as a key pro-apoptotic protein and therefore as a common marker of apoptosis. It exists as inactive proenzymes that undergo proteolytic processing at conserved aspartic residues to produce 2 subunits, large and small, that dimerize to form the active enzyme. FIG. 6A shows that serum starvation (0% FCS, 24 hours) in cultures of neonatal rat ventricular myocytes (NRVM) causes apoptosis, represented by a marked increase in caspase-3 cleavage.

In order to test whether propargylamine can prevent serum starvation-induced apoptosis in cultured neonatal rat ventricular myocytes, we repeated the serum starvation protocol and 0.1 μM propargyl amine was introduced to the culture medium 1 hour before serum starvation. As shown in FIGS. 6B-6C, propargylamine attenuated serum starvation-induced apoptosis in neonatal rat ventricular myocytes as indicated both by the drug-induced decrease in caspase 3 cleavage (FIG. 6B) and increase in the expression of mitochondrial Bcl-2, known as an anti-apoptotic protein (FIG. 6C).

### Example 6

### S(−)-N-propargyl-1-aminoindan Improves Cardiac Function

As the first step in testing the beneficial in vivo efficacy of the propargylamine derivatives on the cardiac function, we measured key cardiovascular hemodynamic parameters in control native rats, and in rats administered IV with a bolus of 1 mg/kg S(−)-N-propargyl-1-aminoindan, followed with a bolus of 10 mg/kg S(−)-N-propargyl-1-aminoindan (Sprague Dawley rats were used, n=3 rats in each group). Measurements were made at baseline, 30 minutes after each drug administration, and 1 hour (recovery) after drug administration.

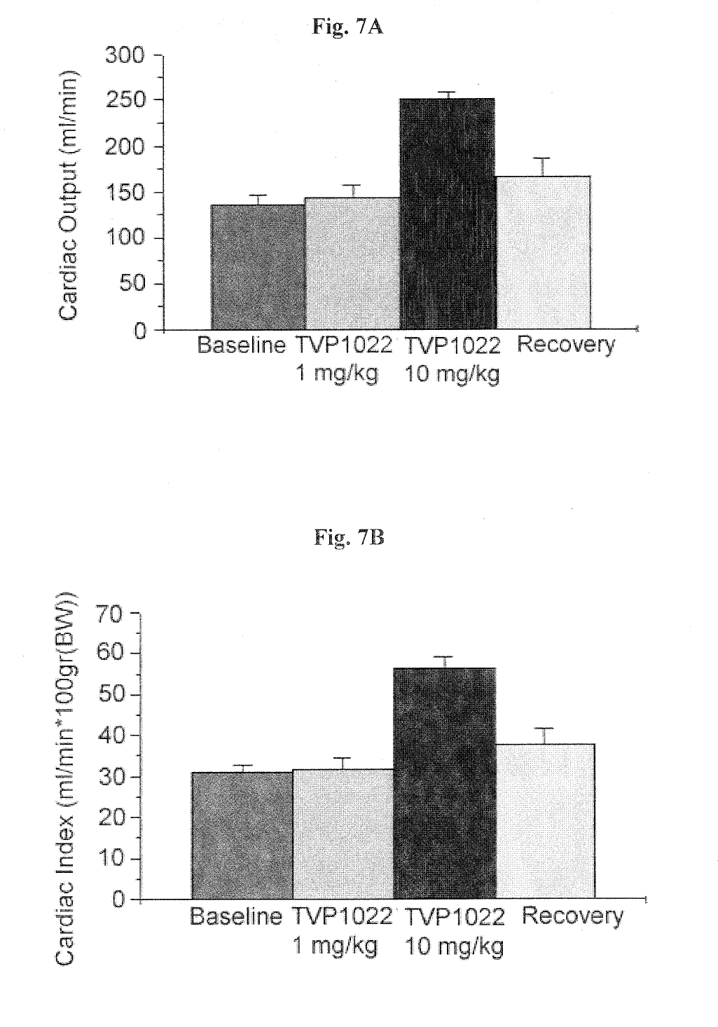

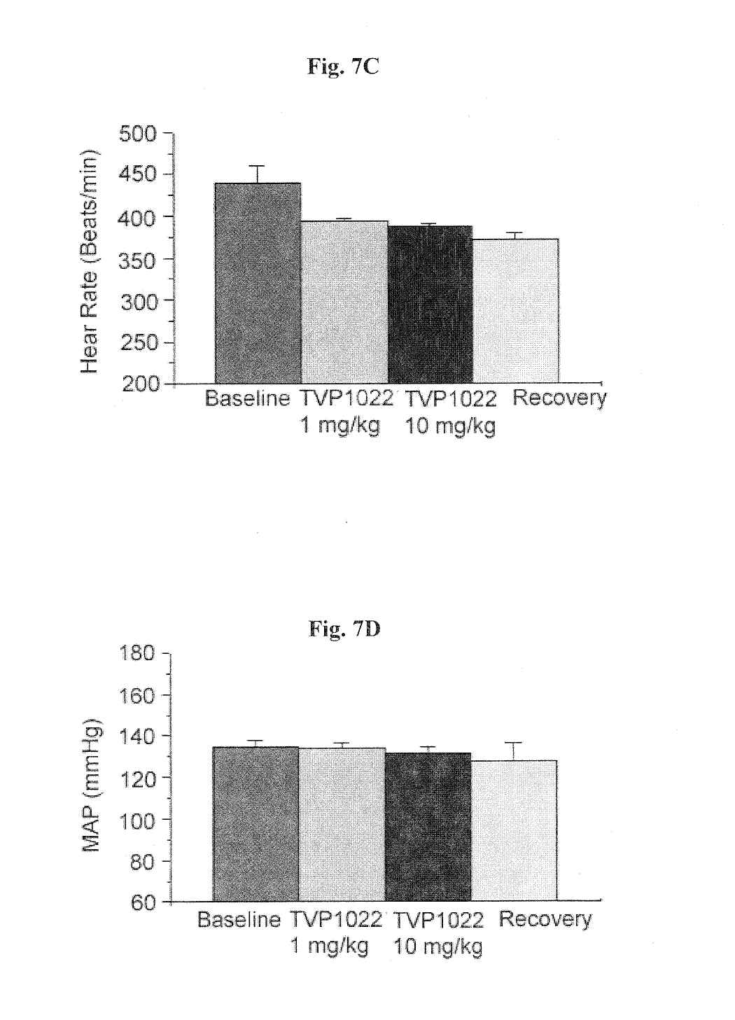

As shown in FIGS. 7A-7D, intravenous administration of 10 mg/kg S(−)-N-propargyl-1-aminoindan had prominent beneficial effect on cardiac function. In particular, S(−)-N-propargyl-1-aminoindan markedly increased cardiac output (7A) and cardiac index (7B), but did not affect heart rate (7C) or mean arterial pressure (MAP) (7D). The above-described effect was reversible during the washout period.

### Example 7

### Propargylamine and S(−)-N-propargyl-1-aminoindan Increase Anti-Apoptotic Proteins in Naïve Rats

The major goal of the experiments described in the following Examples was to examine whether pre-treatment with a propargylamine derivative can confer protection against “future” stressful cardiac insults. The clinical implication of this question is whether it will be able to protect patients at risk. In particular, we investigated whether propargylamine and S(−)-N-propargyl-1-aminoindan can attenuate the cardiac dysfunction in rats with congestive heart failure (CHF) caused by volume overload induced by aortocaval fistula (AVF).

In this experiment we tested the effects of propargylamine and S(−)-N-propargyl-1-aminoindan on several key anti-apoptotic and pro-apoptotic proteins in hearts of naïve rats.

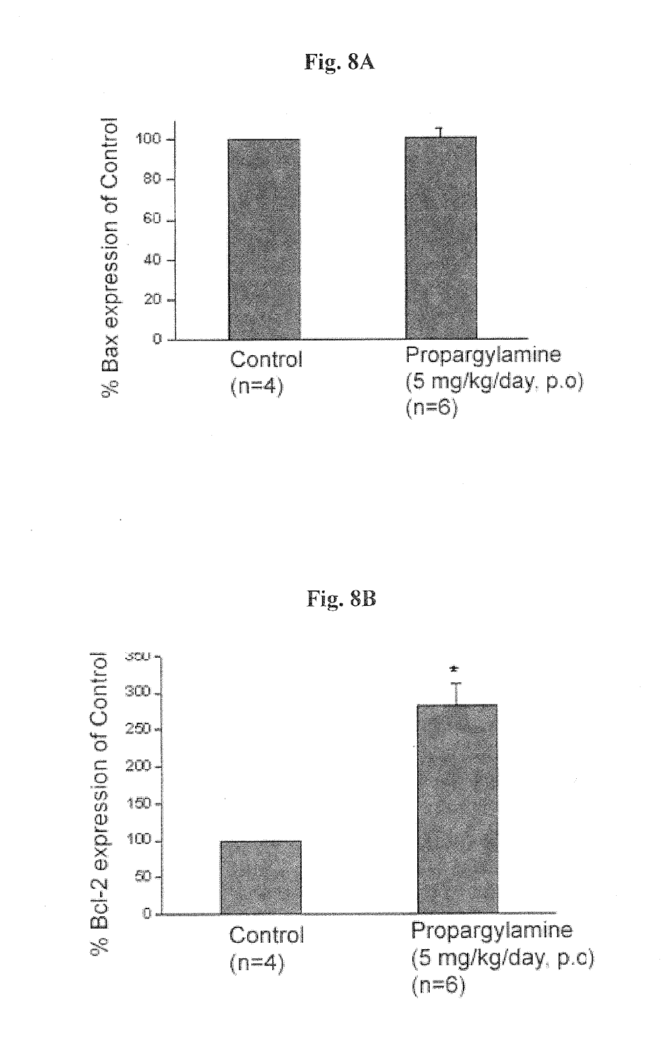

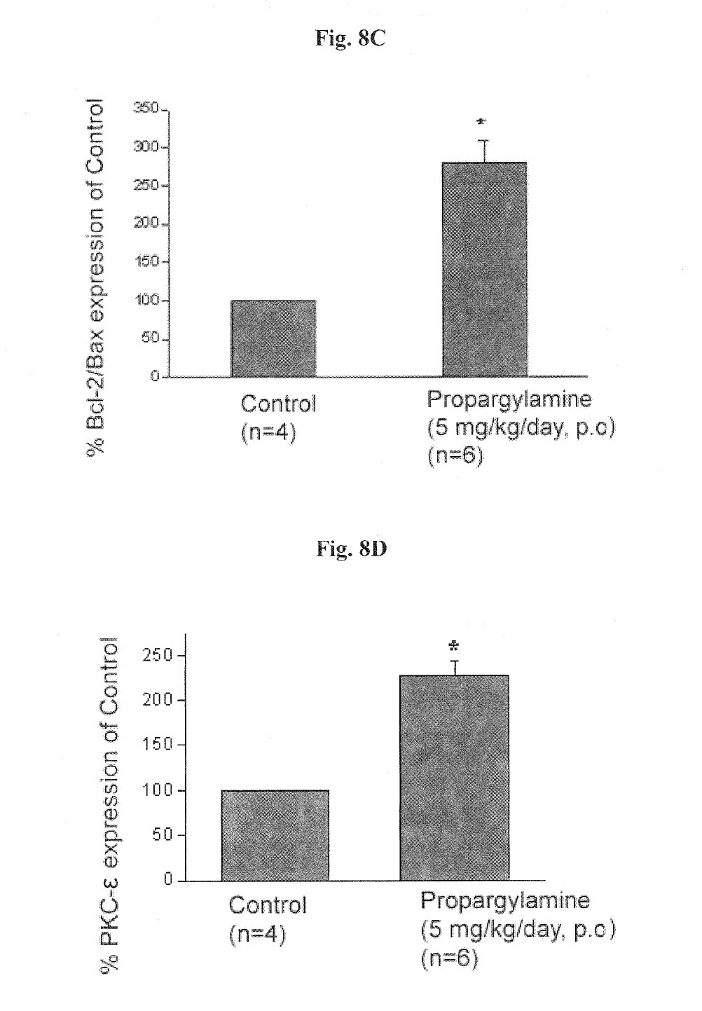

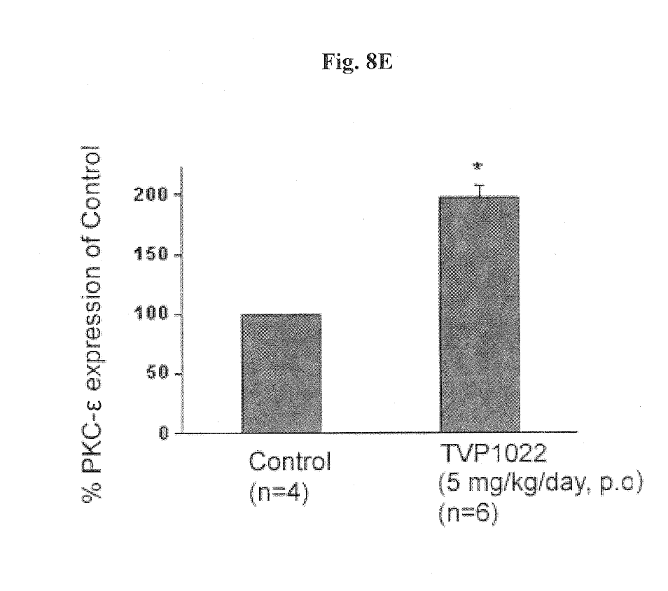

The drugs (5 mg/kg/day) were orally administered to rats for 21 days (n=4-6 rats in each group), and measurements were made after sacrifice. These experiments showed that propargylamine did not affect the expression of mitochondrial pro-apoptotic protein Bax (FIG. 8A), whereas it markedly increased the expression of the mitochondrial anti-apoptotic protein Bcl-2 (FIG. 8B), resulting in marked increase in the ratio Bcl-2/Bax (FIG. 8C), thus generating an anti-apoptotic effect. Furthermore, both propargylamine and S(−)-N-propargyl-1-aminoindan increased the expression of the key anti-apoptotic PKC-ε (FIGS. 8D-8E, respectively).

### Example 8

### Propargylamine and S(−)-N-propargyl-1-aminoindan Generate an Anti-Apoptotic Effect in CHF Rats

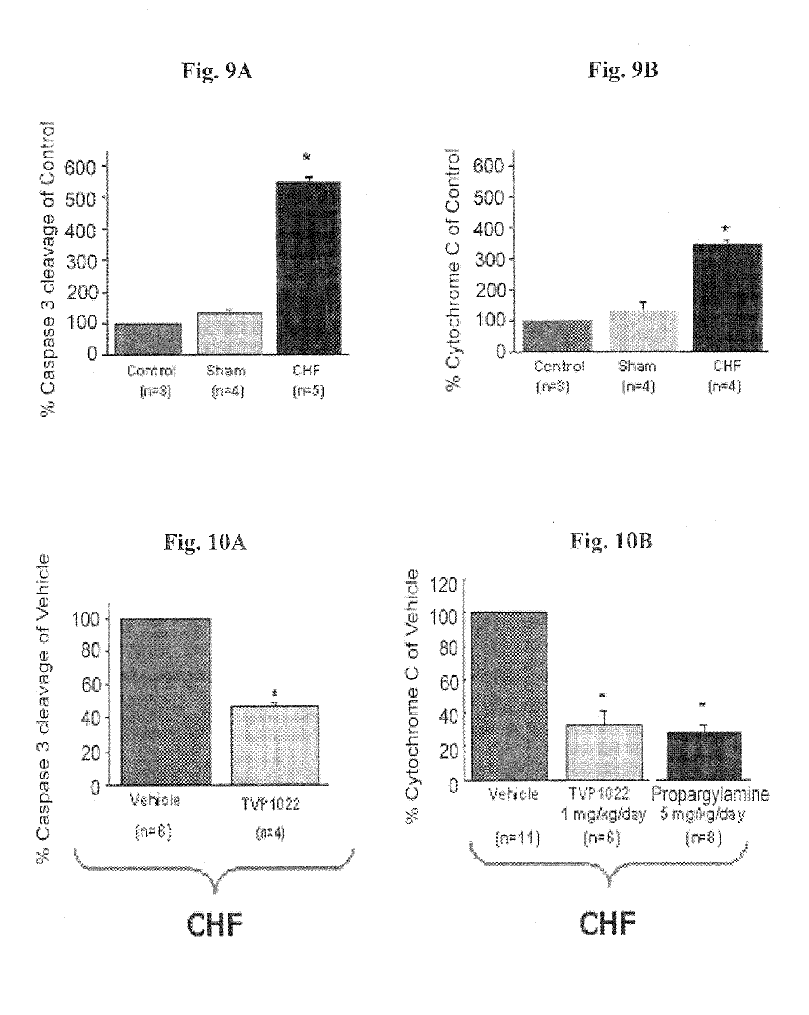

Rats were treated as described in Materials and Methods hereinabove and volume overload was induced by surgical creation of an aortocaval fistula (AVF). Sham-operated rats served as controls. 14 days after induction of volume-overload, caspase-3 cleavage and cytosolic cytochrome C, both pro-apoptotic proteins, were analyzed. As shown in FIGS. 9A-9B, both caspase-3 and cytochrome C were markedly increased, indicating that volume overload-induced congestive heart failure (CHF) is associated with increased expression of these two proteins.

In the following experiment we tested whether propargylamine or S(−)-N-propargyl-1-aminoindan can reduce CHF-induced increase in caspase-3 and cytochrome C. Rats were treated and drugs were administered (1 or 7.5 mg/kg/day S(−)-N-propargyl-1-aminoindan, or 5 mg/kg/day propargylamine) as described in Materials and Methods hereinabove. As shown in FIGS. 10A-10B, both drugs significantly reduced CHF-induced increase in caspase-3 and cytochrome C, suggesting that propargylamine derivatives produce an anti-apoptotic effect both in control and CHF rats, by shifting the balance between the anti-apoptotic proteins and the pro-apoptotic proteins towards the former.

### Example 9

### Propargylamine and S(−)-N-propargyl-1-aminoindan Prevent Ventricular Hypertrophy and the Decline Ventricular Function in CHF Rats

In this set of experiments we determined the ability of pre-treatment with propargylamine or S(−)-N-propargyl-1-aminoindan to prevent ventricular hypertrophy and the decline in ventricular function in CHF rats.

Rats were treated as described in Materials and Methods hereinabove and volume overload was induced by surgical creation of an aortocaval fistula (AVF). Drugs (7.5 mg/kg/day) were administered according to the protocol described above, starting 7 days prior to surgery (day 0) and during 21 days. Cardiac function was determined by echocardiography, from which two principle parameters, namely, diastolic area and systolic area, were calculated. These parameters were used for calculating the fractional shortening, which is an established measure of the ventricular contraction capacity, according to the equation: Fractional shortening=(diastolic area−systolic area)/diastolic area.

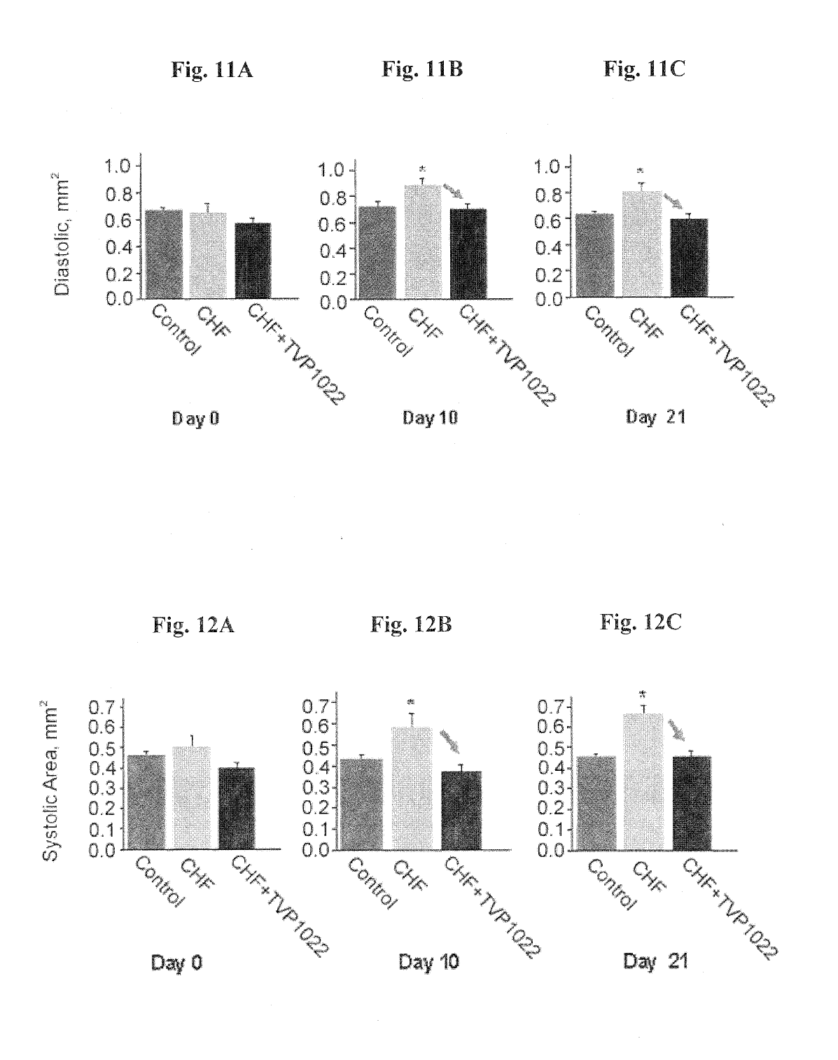

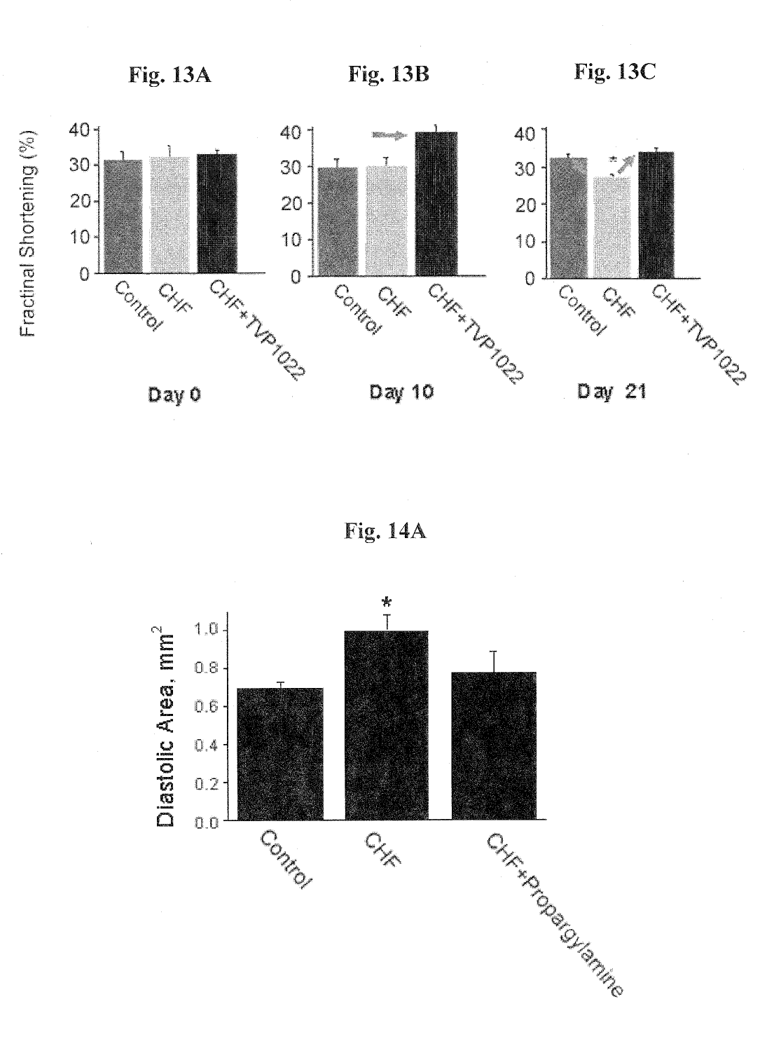

As shown in FIGS. 11 and 12, respectively, the treatment with S(−)-N-propargyl-1-aminoindan completely prevented the hypertrophic increase in the diastolic and systolic areas seen in the CHF group (n=3) at days 10 (3 days post-surgery) and 21 (14 days post-surgery). Furthermore, as shown in FIG. 13, the fractional shortening in the CHF rats on day 21 was significantly reduced compared to the control rats, but S(−)-N-propargyl-1-aminoindan completely prevented this reduction.

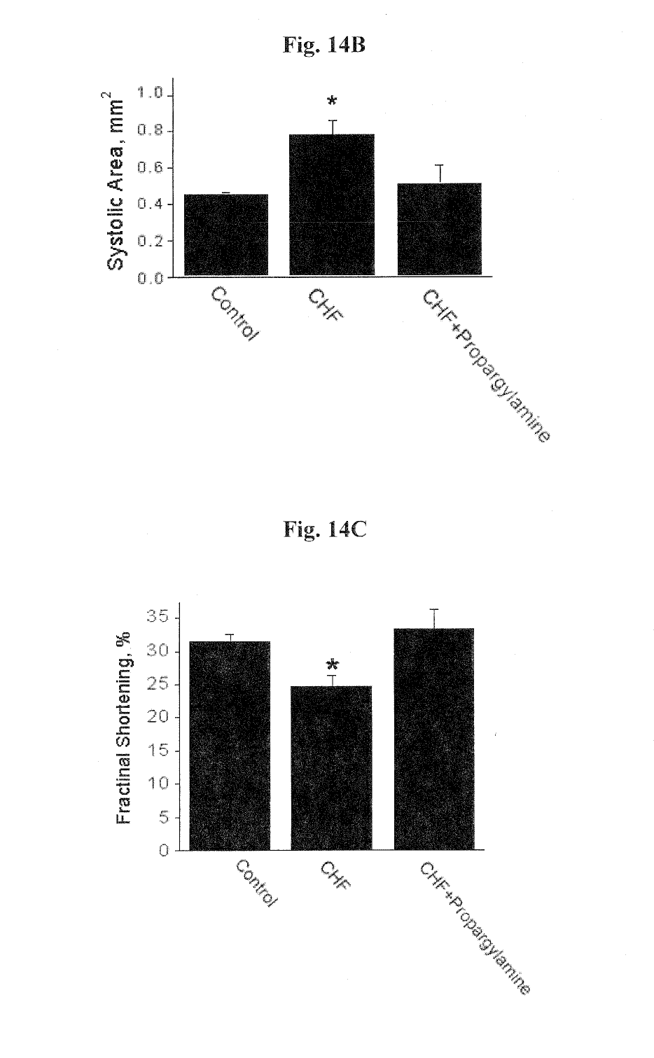

Similar results were obtained with propargylamine using identical experimental and drug administration protocols. As shown in FIGS. 14A-14B, the treatment with propargylamine completely prevented the hypertrophic increase in the diastolic and systolic areas seen in the CHF rats 14 days post-surgery. FIG. 14C shows that the fractional shortening in the CHF rats, 14 days post-surgery was significantly reduced; however, this reduction was completely prevented by the propargylamine.

These in vivo experiments are of prime importance since they demonstrate that both S(−)-N-propargyl-1-aminoindan and propargylamine block the volume-overload induced hypertrophy and the reduction in ventricular mechanical function in CHF rats.

### Example 10

### S(−)-N-propargyl-1-aminoindan Protects Cultured Neonatal Rat Ventricular Myocytes Against Doxorubicin-Induced Apoptosis

Doxorubicin (adriamycin) is a commonly used, highly effective anti cancer drug. However, its clinical efficacy is limited by severe acute cardiotoxic effects, e.g., apoptosis, which limit the total dose of the medicine that may be used safely.

In this set of experiments, we characterized the cardiotoxic effects of doxorubicin in neonatal rat ventricular myocytes (NRVM) by (i) visualizing the nuclear morphology for measuring percent of apoptotic myocytes; and (ii) determining the effect of doxorubicin on the expression of the common apoptotic markers cleaved caspase 3, Bcl-2 and Bax (Puthalakath et al. 1999). Cultures were pretreated with S(−)-N-propargyl-1-aminoindan (1 μM) for 24 hrs before exposure to doxorubicin (0.5 μM) for additional 24 hrs. Apoptotic nuclei were determined by DAPI staining assay, and cleaved caspase-3, Bcl-2 and Bax were determined by means of immunoblotting analysis in cell lysates, both methods are described in Materials and Methods. All the results are expressed as fold of control levels (untreated cultures). Loading of the lanes was normalized to β-actin levels.

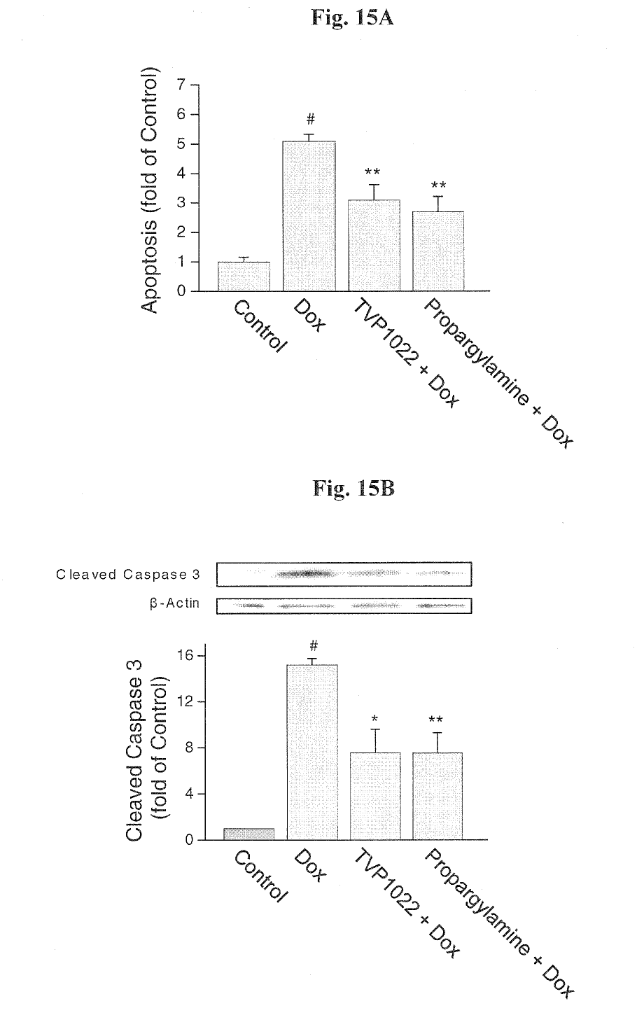

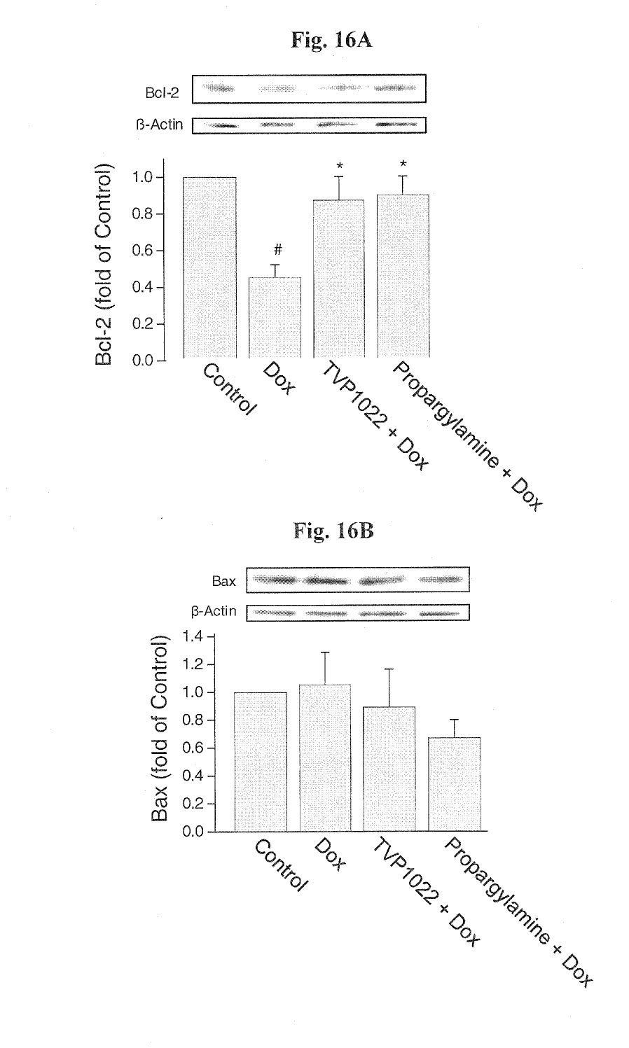

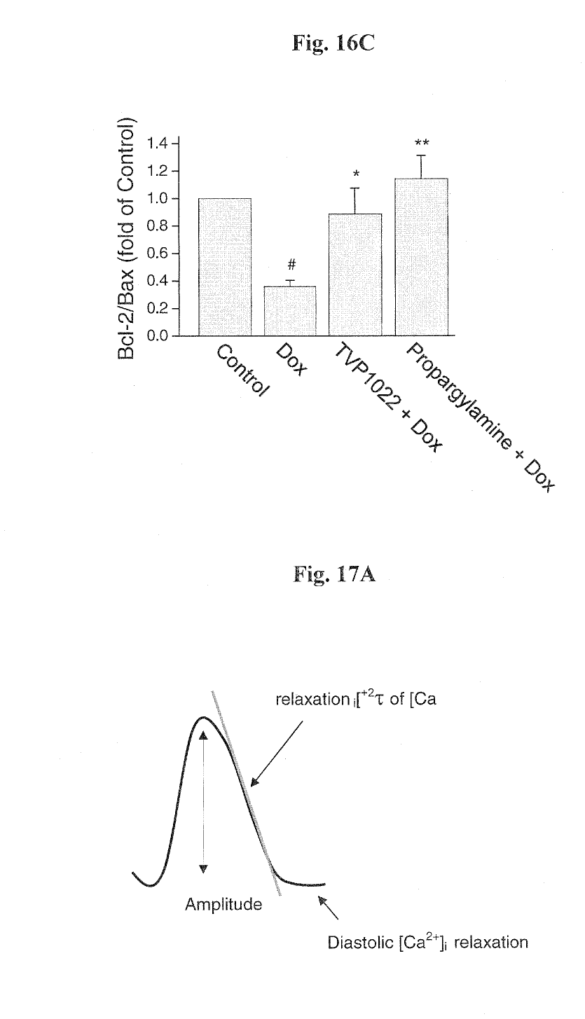

As shown in FIG. 15A, incubation of NRVM with doxorubicin for 24 hrs caused a ˜5-fold increase (P<0.001) in myocytes' apoptosis, as previously described (Jeremias et al., 2005; Kunisada et al., 2002; Li et al., 2006; Ueno et al., 2006; Wu et al. 2002). In agreement with this finding, doxorubicin increased cleaved caspase 3 expression by ˜14 fold, P<0.001 (FIG. 15B) and decreased Bcl-2 expression (FIG. 16A) without changing Bax expression (FIG. 16B), thus decreasing Bcl-2/Bax ratio by ˜50% (FIG. 16C).

In order to determine whether S(−)-N-propargyl-1-aminoindan can attenuate doxorubicin-induced apoptosis, NRVM were treated with the neuroprotective concentration of the drug (1 μM) (Maruyama et al., 2001) for 24 hrs prior to adding doxorubicin. As depicted in FIG. 15A, S(−)-N-propargyl-1-aminoindan significantly (P<0.01) attenuated doxorubicin-induced apoptosis. Accordingly, S(−)-N-propargyl-1-aminoindan inhibited doxorubicin-induced increase in cleaved caspase 3 (FIG. 15B), and prevented the decrease in Bcl-2 levels (FIG. 16A), thus completely prevented the reduction in doxorubicin-induced Bcl-2/Bax ratio (FIG. 16C). S(−)-N-propargyl-1-aminoindan did not affect control NRVM in the absence of doxorubicin (data not shown).

### Example 11

### S(−)-N-propargyl-1-aminoindan Attenuates the Deleterious Effects of Doxorubicin on the [Ca²⁺]ᵢ Transient and Contraction of NRVM

In addition to its apoptotic effect, doxorubicin was previously shown to affect the [Ca²⁺]ᵢ transients and contractions of NRVM (Fixler et al., 2002; Maeda et al., 1999; Mijares and Lopez, 2001; Shneyvays et al., 2001; Timolati et al., 2006; Wang et al., 2001).

Cultures were pre-incubated with S(−)-N-propargyl-1-aminoindan (1 μM) for 24 hrs before adding doxorubicin (0.5 μM) for additional 24 hrs. The [Ca²⁺]ᵢ transient parameters and myocytes contraction properties were measured and determined as described in Materials and Methods.

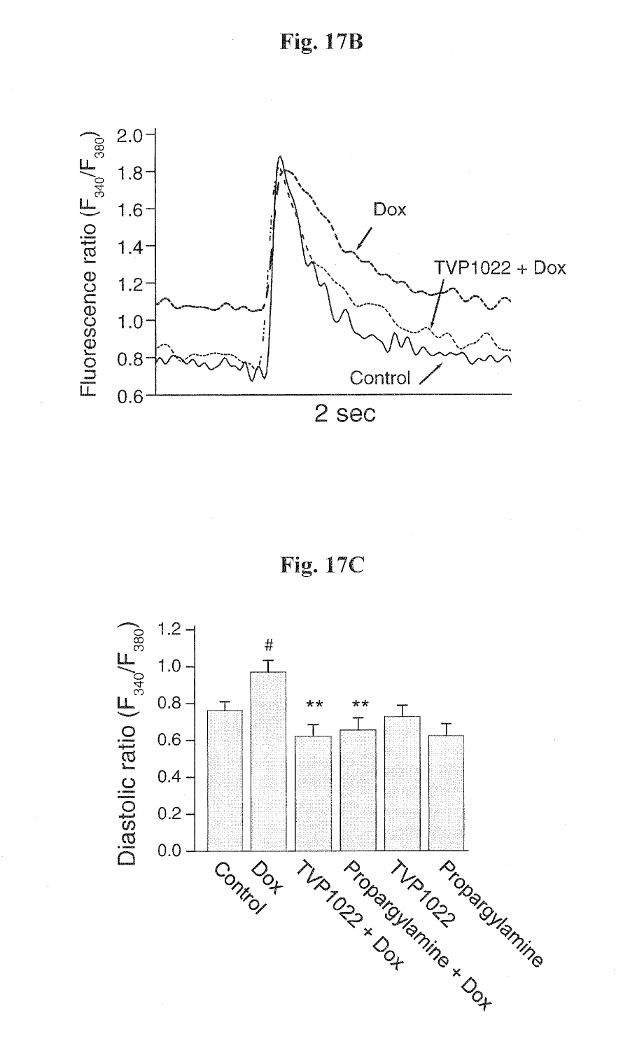

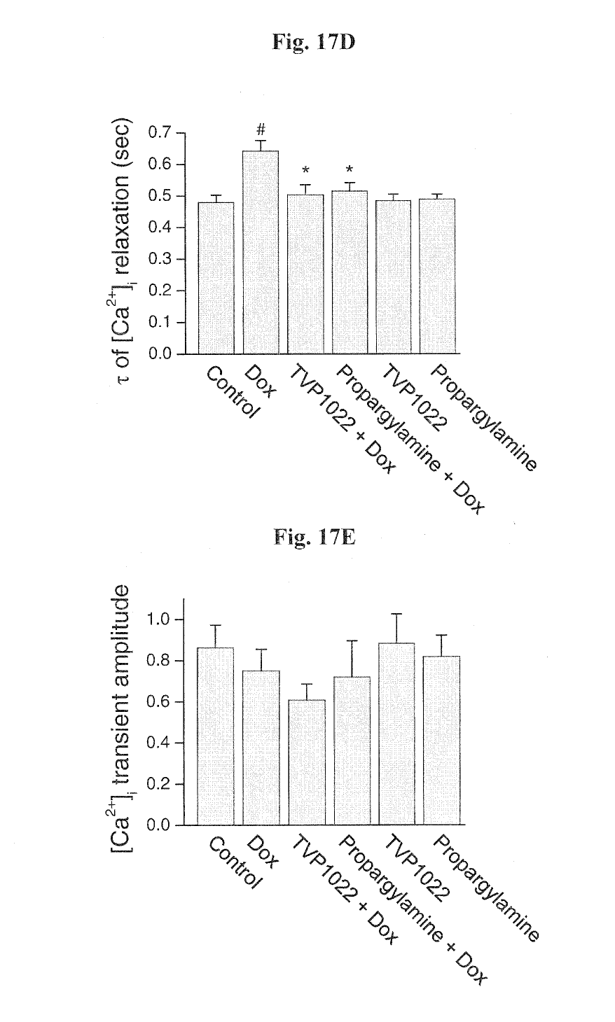

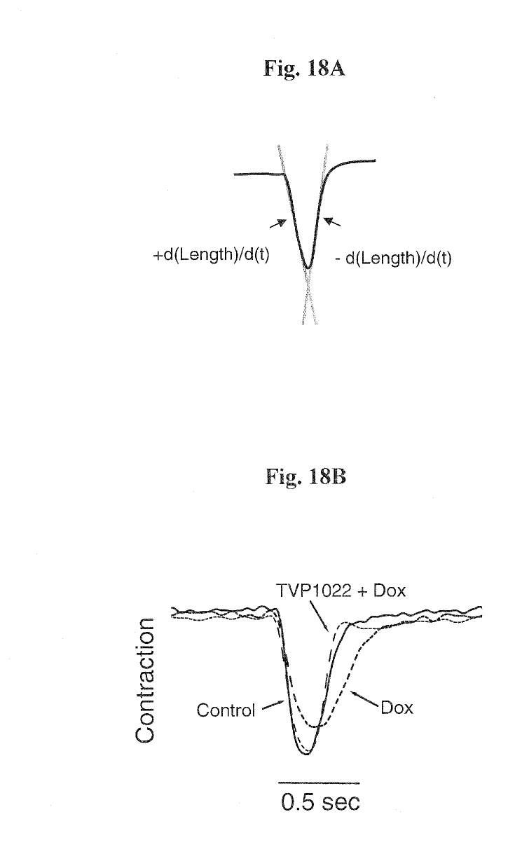

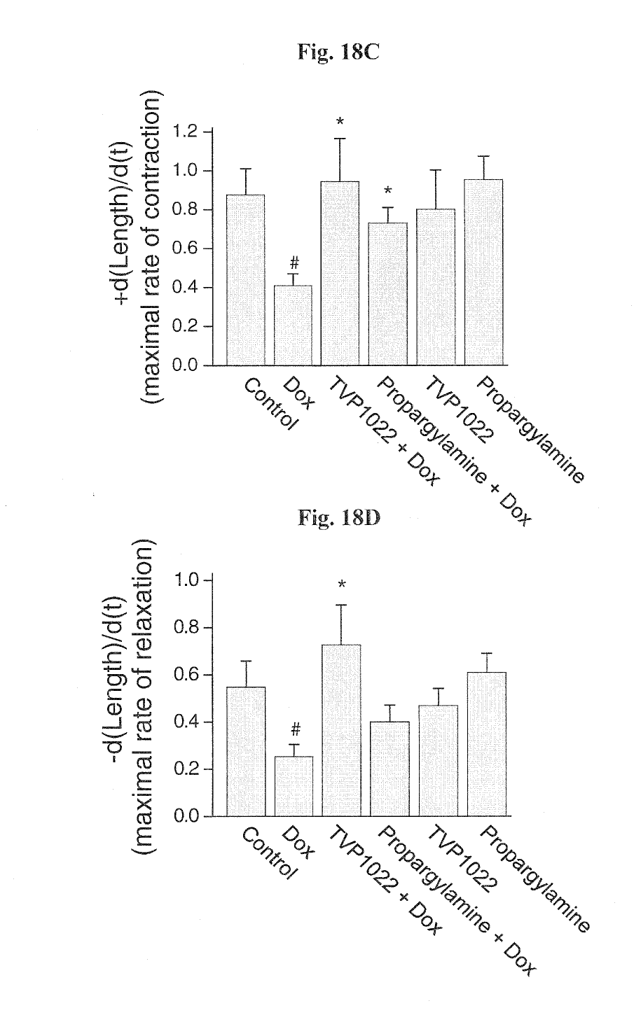

As depicted by a representative experiment (FIG. 17B) and by the summary of five experiments, incubation of NRVM with doxorubicin for 24 hrs elevated (P<0.01) diastolic [Ca²⁺]_{I} and slowed (P<0.01) the kinetics of the [Ca²⁺]ᵢ transient relaxation, as shown in FIGS. 17C and 17D, respectively; however, did not affect [Ca²⁺]ᵢ transient amplitude (data not shown). As expected, doxorubicin also affected the contraction properties, decreasing the maximal rates of contraction and relaxation (P<0.05), as shown in FIGS. 18B-18D.

In agreement with its anti-apoptotic effects, S(−)-N-propargyl-1-amino indan completely prevented the deleterious effects of doxorubicin on the [Ca²⁺]ᵢ transients and contractions of NRVM, as shown in FIGS. 17B-17E and in FIGS. 18B-18D, respectively. Importantly, and as shown in these Figures, S(−)-N-propargyl-1-aminoindan did not affect the [Ca²⁺]ᵢ transient or the contraction parameters in control NRVM. These latter findings are of particular importance due to the potential therapeutic efficacy of S(−)-N-propargyl-1-aminoindan.

### Example 12

### The Effects of Propargylamine on Doxorubicin-Induced Apoptosis, [Ca²⁺]ᵢ Transients and Contraction of NRVM

In order to determine the importance of the propargyl moiety in the cardioprotective activity of S(−)-N-propargyl-1-aminoindan, we investigated the ability of propargylamine to attenuate doxorubicin-induced apoptosis, and deleterious effects of doxorubicin on the [Ca²⁺]ᵢ transient and contraction of NRVM. Cultures were pretreated with propargylamine (1 μM) for 24 hrs before exposure to doxorubicin (0.5 μM) for additional 24 hrs. Apoptotic nuclei were determined by DAPI staining assay, and cleaved caspase-3, Bcl-2 and Bax were determined by means of immunoblotting analysis in cell lysates. The [Ca²⁺]ᵢ transient parameters and myocytes contraction properties were measured and determined as described in Materials and Methods.

As depicted in FIGS. 15A-15B, propargylamine reduced doxorubicin-induced apoptosis (P<0.01) and cleaved caspase 3 level (P<0.01). Accordingly, and similar to S(−)-N-propargyl-1-aminoindan, propargylamine increased Bcl-2 expression (P<0.05) (FIG. 16A), thus completely prevented doxorubicin-induced decrease in Bcl-2/Bax ratio (FIG. 16C). Similar to S(−)-N-propargyl-1-aminoindan, propargylamine prevented doxorubicin-induced increase in diastolic [Ca²⁺]ᵢ (P<0.01), the decrease in the rate of [Ca²⁺]ᵢ relaxation (P<0.05), as well as the reduction in the maximal rate of contraction (P<0.05). Furthermore, propargylamine did not inhibit doxorubicin-induced decrease in the maximal rate of relaxation, and like S(−)-N-propargyl-1-aminoindan, it did not affect the [Ca²⁺]ᵢ transient or the contraction parameters in control NRVM, as shown in FIGS. 17C-17E and 18C-18D, respectively.

### Example 13

### S(−)-N-propargyl-1-aminoindan and propargylamine do not Cause Human Cancer Cell Proliferation and do not Affect the Anti-Cancer Effect of Doxorubicin in Human Cancer Cells

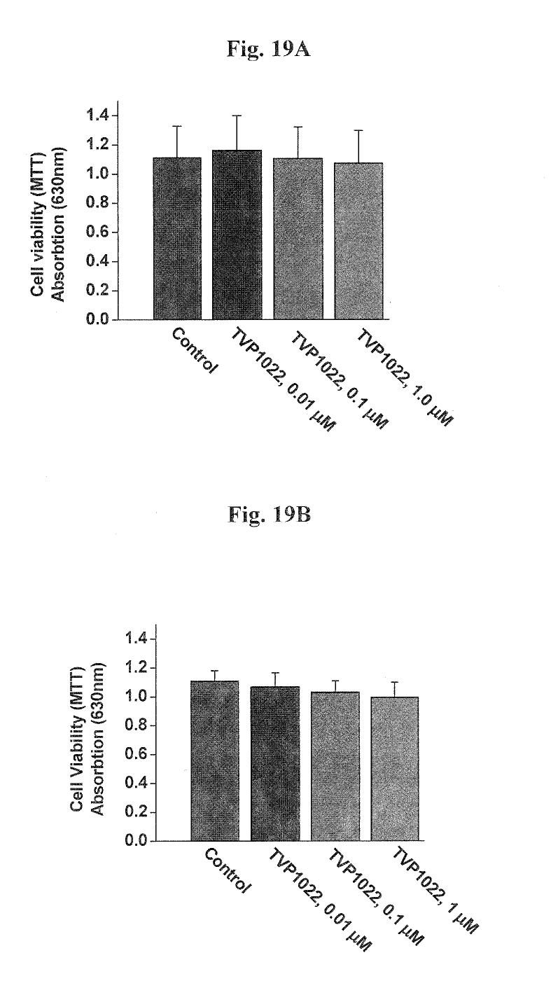

Since S(−)-N-propargyl-1-aminoindan is considered to be administered to cancer patients, in this experiment we first examined whether due to its anti-apoptotic effect it will enhance proliferation of cancer cells. For this purpose, human cervical carcinoma HeLa and breast carcinoma MDA-231 cells were incubated with or without S(−)-N-propargyl-1-aminoindan (0.01, 0.1 or 1 μM), for 48 hrs, and cell proliferation was determined by the MTT staining assay as described in Material and Methods. As shown in FIGS. 19A-B, S(−)-N-propargyl-1-aminoindan, at each one of the concentrations tested, did not cause cancer cells proliferation both in HeLa as well as in MDA-231 cells.

In view of the effects of both S(−)-N-propargyl-1-aminoindan and propargylamine on doxorubicin-induced apoptosis, [Ca²⁺]ᵢ transients and contraction of NRVM, described in Examples 10-12 hereinabove, we investigated whether these two active agents interfere with the anti-cancer activity of doxorubicin in various human cancer cell lines.

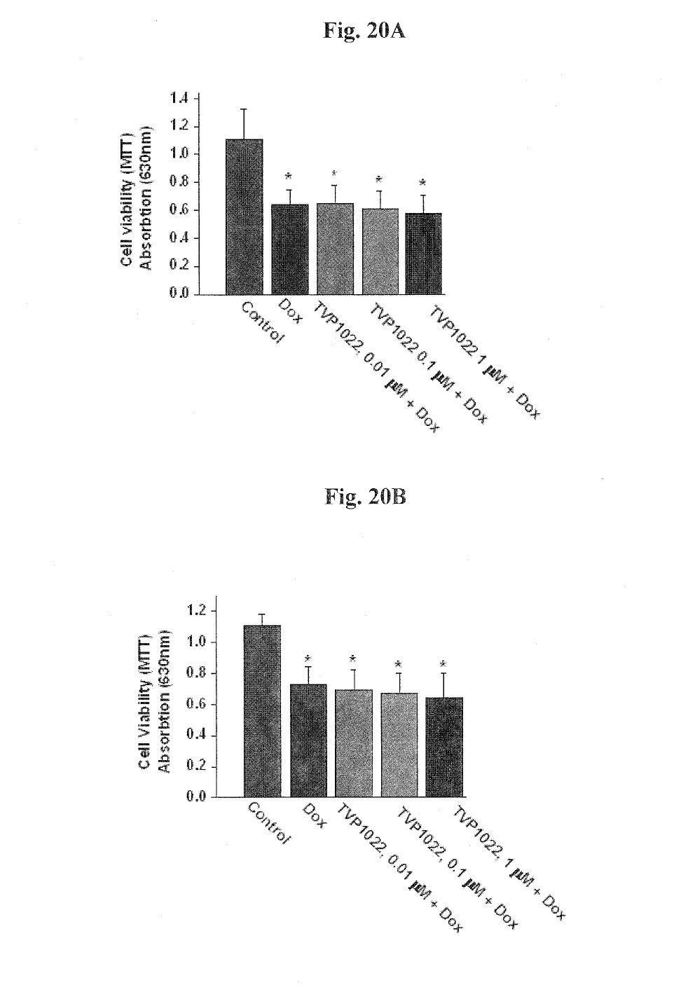

In the first experiment, human cervical carcinoma HeLa and breast carcinoma MDA-231 cells were pre-incubated with or without S(−)-N-propargyl-1-aminoindan (0.01, 0.1 or 1 μM) for 24 hrs, and then treated with doxorubicin (1 μM in the case of HeLa cells and 10 μM in the case of MDA-231 cells) for additional 24 hrs. Cell viability was determined by the MTT staining assay as described in Material and Methods. As expected, cellular viability of the two cancer cell lines was markedly reduced by doxorubicin; however, with respect to both cancer cell lines tested, none of the S(−)-N-propargyl-1-aminoindan concentrations affected doxorubicin-induced cancer cell death, as shown in FIGS. 20A-B.

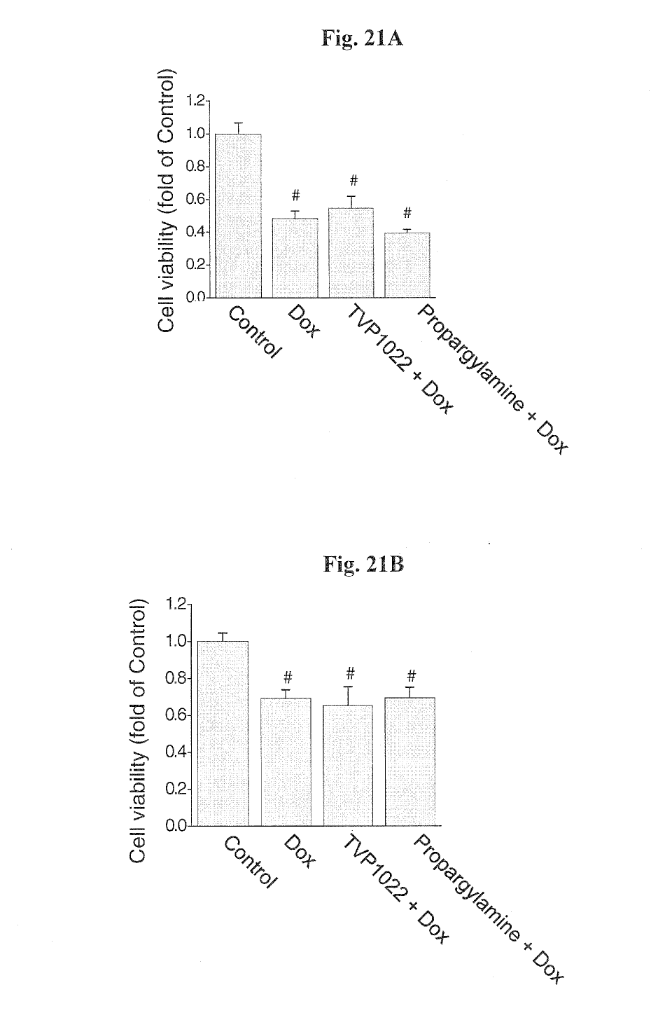

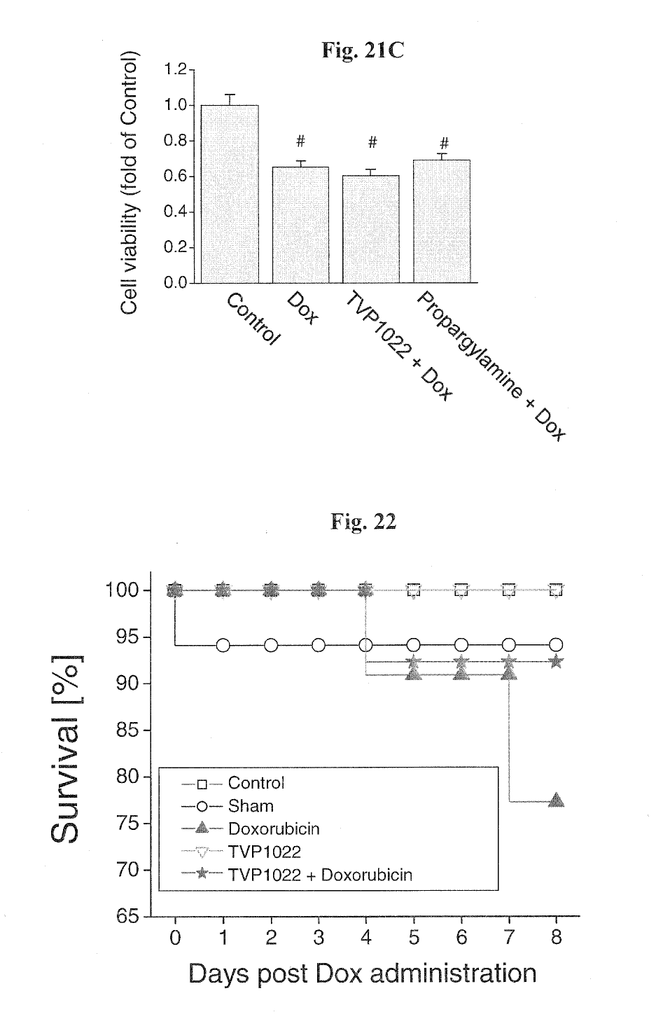

In the second experiment, both S(−)-N-propargyl-1-aminoindan and propargylamine, at a concentration of 1 μM, were tested, using human cervical carcinoma HeLa, breast carcinoma MDA-231 and breast carcinoma MDA-415 cell lines. The various cancer cells were pre-incubated with or without S(−) -N-propargyl-1-aminoindan or propargylamine for 24 hrs, and then treated with doxorubicin (10 μM) for additional 24 hrs. Cell viability was determined by the MTT staining assay. Similarly to the first observation, cellular viability of all three cancer cell lines was markedly reduced by doxorubicin (˜30-50%); however, with respect to all human cancer cell lines tested, neither S(−)-N-propargyl-1-aminoindan nor propargylamine affected doxorubicin-induced cancer cell death, as shown in FIGS. 21A-C, indicating that pretreatment and co-administration of S(−)-N-propargyl-1-aminoindan or propargylamine do not interfere with the anti-cancer efficacy of doxorubicin.

### Example 14

### S(−)-N-propargyl-1-aminoindan Increases Survival of Doxorubicin-Treated Mice and Prevents Doxorubicin-Induced Decrease in Body and Heart Weight

In this set of experiments we determined the ability of pre-treatment with S(−)-N-propargyl-1-aminoindan to increase the survival of doxorubicin-treated mice.

Mice were treated as described in Materials and Methods hereinabove. In particular, mice were divided into 5 experimental groups, wherein mice in the doxorubicin group were IV injected with one dose of doxorubicin, 20 mg/kg, into the tail vein (n=22); (ii) mice in the control group were untreated (n=9); mice in the sham group were fed with DDW and injected with doxorubicin vehicle (saline) (n=17); mice in the TVP1022 group were fed with TVP1022, 7.5 mg/kg/day, for 15 days (n=13); and mice in the TVP1022+doxorubicin group were fed with TVP1022, 7.5 mg/kg/day, for 15 days, and on day 7 were IV injected with doxorubicin, 20 mg/kg, into the tail vein (n=13).

The first sign of doxorubicin toxicity was apparent 5-7 days post-injection, wherein the mice injected with doxorubicin were less active than the mice of the other groups, including the mice of the TVP1022+doxorubicin group. In particular, the doxorubicin-injected mice tended to stand on one spot, while the rest of the animals moved around vividly in the cage.

As shown in FIG. 22, pretreatment with TVP1022 decreased the mortality, namely, increased the survival, of doxorubicin-treated mice. In particular, mortality was decreased from 22.7% (5/22 mice) in the doxorubicin-treated group, at day 8 after doxorubicin administration, to 7.6% (1/13 mice) in the TVP1022+doxorubicin group, as in the saline-injected mice (5.9%).

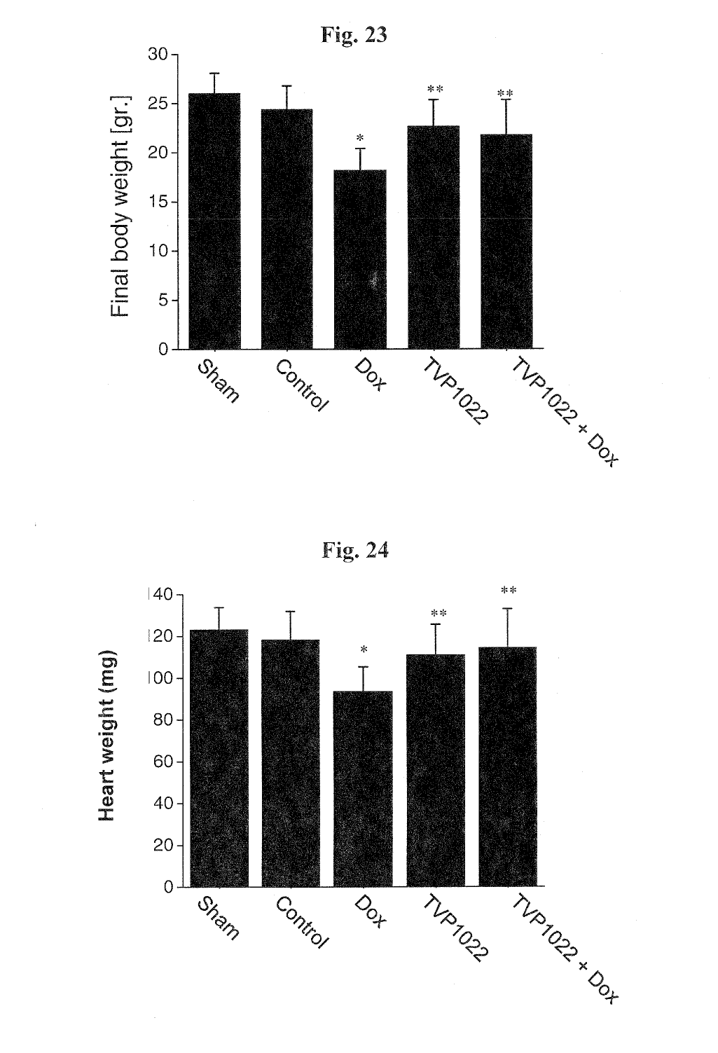

FIG. 23 and FIG. 24 show the average final body weight and the average heart weight respectively, as measured in the surviving animals of each one of the various groups. As shown in these Figures, the final body weight of the doxorubicin-treated group (18.2±2 gr) was significantly (P<0.01) lower than the final body weight of the control group (24.4±2.4 gr), the sham group (26-2 gr), the TVP1022 group (22.6±2.7 gr) and the TVP1022+doxorubicin group (21.8±3.6 gr); and the heart weight of the doxorubicin-treated group (93.5±12 mg) was significantly (P<0.001) lower than the heart weight of the control group (123±11 mg), the sham group (118±14 mg), the TVP1022 group (111±15 mg) and the TVP1022+doxorubicin group (114±19 mg).

### REFERENCES

Abu-Raya, S. Tabakman, R. Blaugrund, E. Trembovler, V. Lazarovici, P., Neuroprotective and neurotoxic effects of monoamine oxidase-B inhibitors and derived metabolites under ischemia in PC12 cells, Eur. J. Pharmacol., 2002, 434, 109-116
Akao, Y. Maruyama, W. Hong, Y. Shamoto-Nagai, M. Youdim, M. B. H. Naoi, M., An anti-Parkinson's disease drug, N-propargyl-1(R)-aminoindan (rasagiline), enhances expression of anti-apoptotic Bcl-2 in human dopaminergic SH-SY5Y cells, Neurosci. Lett., 2002, 326, 105-108
Am, O. B. Amit, T. Youdim, M. B. H., Contrasting neuroprotective and neurotoxic actions of respective metabolites of anti-Parkinson drugs rasagiline and selegiline, Neurosci. Lett., 2004, 355, 169-172
Arai, M. Tomaru, K. Takizawa, T. Sekiguchi, K. Yokoyama, T. Suzuki, T. Nagai, R., Sarcoplasmic reticulum genes are selectively down-regulated in cardiomyopathy produced by doxorubicin in rabbits, J. Mol. Cell. Cardiol., 1998, 30, 243-254
Arai, M. Yoguchi, A. Takizawa, T. Yokoyama, T. Kanda, T. Kurabayashi, M. Nagai, R., Mechanism of doxorubicin-induced inhibition of sarcoplasmic reticulum Ca²⁻-ATPase gene transcription, Circ. Res., 2000, 86, 8-14
Birkmayer, W. Riederer, P. Ambrozi, L. Youdim, M. B. H., Implications of combined treatment with madopar and 1-deprenyl in Parkinson's disease, Lancet., 1977, 2, 439-443

Brooks, D. J. Sagar, H., UK-irish Entacapone Study Group, Entacapone is beneficial in both fluctuating and non-fluctuating patients with Parkinson's disease: a randomized, placebo controlled, double blind, six month study, J. Neurol. Neurosurg. Psychiatry, 2003, 74, 1071-1079
Clerk, A. Cole, S. M. Cullingford, T. E. Harrison, J. G. Jormakka, M. Valks, D. M., Regulation of cardiac myocyte cell death. Pharmacol. Ther., 2003, 97, 223-261
Doroshow, J. H., Anthracyclines and anthracenediones, 1996, p. 500-537 In Chabner, B. A. Longo, D. L. (ed.), Cancer chemotherapy and biotherapy: principles and practice, 2001, 3^{rd} ed., Lippincott Williams and Wilkins, Philadelphia, Pa.
Durden, D. A. Dyck, L. E. Davis, B. A. Liu, Y. D. Boulton, A. A., Metabolism and pharmacokinetics, in the rat, of (R)—N-(2-Heptyl)Methyl-propargylamine (R-2HMP), a new potent monoamine oxidase inhibitor and antiapoptotic agent, Drug Metab. Dispos., 2000, 28(2), 147-154
Feng, W. Liu, G. Xia, R. Abramson, J. J. Pessah, I. N. Site-selective modification of hyperreactive cysteines of ryanodine receptor complex by quinones, Mol Pharmacol, 1999, 55, 821-831
Finberg, J. P. Ari, G. Lavian, G. Hovevey-Sion, D., Modification of alpha-2 presynaptic receptor activity and catecholamine release following chronic MAO inhibition, J Neural Transm Suppl., 1990, 32, 405-412
Finberg, J. P. M. Lamensdorf, I. Weinstock, M. Schwartz, M. Youdim, M. B. H., Pharmacology of rasagiline (N-propargyl-IR-aminoindan), Adv. Neurol., 1999, 80, 495-501
Fixler, D. Tirosh, R. Zinman, T. Shainberg, A. Deutsch, M., Differential aspects in ratio measurements of [Ca²⁺¹]ᵢ relaxation in cardiomyocyte contraction following various drug treatments, Cell Calcium, 2002, 31, 279-287
Friberg, H. Wicloch, T., Mitochondrial permeability transition in acute neurodegeneration, Biochimie, 2002, 84, 241-250
Glezer, S. Finberg, J. P., Pharmacological comparison between the actions of methamphetamine and 1-aminoindan stereoisomers on sympathetic nervous function in rat vas deferens, Eur. J. Pharmacol., 2003, 472, 173-177
Green, P. S. Leeuwenburgh, C., Mitochondrial dysfunction is an early indicator of doxorubicin-induced apoptosis, Biochim. Biophys. Acta, 2002, 1588, 94-101

Halestrap, A. P. Clarke, S. J. Javadov, S. A., Mitochondrial permeability transition pore opening during myocardial reperfusion—a target for cardioprotection, Cardiovasc. Res., 2004, 61, 372-385
Jeremias, I. Stahnke, K. Debatin, K. M., CD95/Apo-1/Fas: independent cell death induced by doxorubicin in normal cultured cardiomyocytes, Cancer Immunol. Immunother., 2005, 54, 655-662
Kalyanaraman, B. Joseph, J. Kalivendi, S. Wang, S. Konorev. E. Kotamraju, S., Doxorubicin-induced apoptosis: implications in cardiotoxicity, Mol. Cell Biochem., 2002, 234-235, 119-124
Kitani, K. Minami, C. Maruyama, W. Kanai, S. Ivy, G. O. Carrillo, M. C., Common properties for propargylamines of enhancing superoxide dismutase and catalase activities in the dopaminergic system in the rat: implications for the life prolonging effect of (−)deprenyl, J. Neural. Transm. Suppl, 2000, 139-156
Kotamraju, S. Konorev, E. A. Joseph, J. Kalyanaraman, B., Doxorubicin-induced apoptosis in endothelial cells and cardiomyocytes is ameliorated by nitrone spin traps and ebselen. Role of reactive oxygen and nitrogen species, J. Biol. Chem., 2000, 275, 33585-33592
Kraemer, T. Maurer, H. H., Toxicokinetics of amphetamines: metabolism and toxicokinetic data of designer drugs, amphetamine, methamphetamine, and their N-alkyl derivatives. Ther. Drug. Monit., 2002, 24, 277-289
Kunisada, K. Tone, E. Negoro, S. Nakaoka, Y. Oshima, Y. Osugi, T. Funamoto, M. Izumi, M. Fujio, Y. Hirota, H. Yamauchi-Takihara, K., Bcl-xl reduces doxorubicin-induced myocardial damage but fails to control cardiac gene downregulation, Cardiovasc. Res., 2002, 53, 936-943
Lavian, G. Finberg, J. P. Youdim, M. B. H., The advent of a new generation of monoamine oxidase inhibitor antidepressants: pharmacologic studies with moclobemide and brofaromnine, Clin. Neuropharmacol., 1993, 16 (Suppl. 2), S1-7
Lees, A. J., Comparison of therapeutic effects and mortality data of levodopa and levodopa combined with selegiline in patients with early, mild Parkinson's disease. Parkinson Disease Research Group of the United Kingdom, Br. Med. J., 1995, 311, 1601-1607
Li, K. Sung, R. Y. T. Huang, W. Z. Yang, M. Pong, N. H. Lee, S. M. Chan, W. Y. Zhao, H. To, M. Y. Fok, T. F. Li, C. K. Wong, Y. O. Ng, P. C., Thrombopoietin protects against in vitro and in vivo cardiotoxicity induced by doxorubicin, Circulation, 2006, 113, 2211-2220
Maeda, A. Honda, M. Kuramochi, T. Takabatake, T., A calcium antagonist protects against doxorubicin-induced impairment of calcium handling in neonatal rat cardiac myocytes, Jpn. Circ. J., 1999, 63, 123-129
Mandel, S. Grunblatt, E. Riederer, P. Gerlach, M. Levites, Y. Youdim, M. B. H., Neuroprotective strategies in Parkinson's disease: an update on progress. CNS. Drugs., 2003, 17, 729-762
Maruyama, W. Akao, Y. Youdim, M. B. Naoi, M., Neurotoxins induced apoptosis in dopamine neurons: protection by N-propargylamine-1(R)- and (S)-aminoindan, rasagiline and TV1022, J. Neural. Transm. Suppl, 2000, 60, 171-186
Maruyama, W. Youdim, M. B. Naoi, M., Antiapoptotic properties of rasagiline, N-propargylamine-1(R)-aminoindan, and its optical (S)-isomer, TV1022, Ann. N Y Acad. Sci., 2001, 939, 320-329
Mattson, M. P. Kroemer, G., Mitochondria in cell death: novel targets for neuroprotection and cardioprotection, Trends Mol. Med., 2003, 9, 196-205
Mijares, A. Lopez, J. R., L-carnitine prevents increase in diastolic [CA²⁻] induced by doxorubicin in cardiac cells, Eur. J. Pharmacol., 2001, 425, 117-120
Olson, R. D. Gambliel, H. A. Vestal, R. E. Shadle, S. E. Charlier, Jr. H. A. Cusack. B. J., Doxorubicin cardiac dysfunction: effects on calcium regulatory proteins, sarcoplasmic reticulum, and triiodothyronine, Cardiovasc. Toxicol., 2005, 5, 269-283
Pollack, M. Phaneuf, S. Dirks, A. Leeuwenburgh, C., The role of apoptosis in the normal aging brain, skeletal muscle, and heart, Ann. N Y Acad. Sci., 2002, 959, 93-107
Puthalakath, H. Huang, D. C. O'Reilly, L. A. King, S. M. Strasser, A., The proapoptotic activity of the Bcl-2 family member Bim is regulated by interaction with the dynein motor complex, Mol. Cell., 1999, 3, 287-296
Reynolds, G. P. Riederer, P. Sandier, N. R. Jellinger, K. Seemali, D., Amphetamine and 2-phenylethylamine in post mortem Parkinson brains after 1-deprenyl administration, J. Neural. Transm., 1978, 43, 271-278
Riederer, P. Rinne, U. K., Selegiline in Parkinson's disease: An update, Mov. Disords., 1992, 8 (Suppl. 1), 51-54
Rubin, Y. Kessler-Icekson, G. Navon, G., The effect of furosemide on calcium ion concentration in myocardial cells, Cell Calcium, 1995, 18, 135-139
Sarvazyan, N., Visualization of doxorubicin-induced oxidative stress in isolated cardiac myocytes, Am. J. Physiol., 1996, 271, H2079-H2085
Sawyer, D. B. Fukazawa, R. Arstall, M. A. Kelly, R. A., Daunorubicin-induced apoptosis in rat cardiac myocytes is inhibited by dexrazoxane, Circ. Res., 1999, 84, 257-265
Shin, H. S., Metabolism of selegiline in humans: Identification, excretion and stereochemistry of urine metabolites, Drug. Metab. Dispos., 1997, 25, 657-662
Shizukuda, Y. Matoba, S. Mian, O. Y. Nguyen, Hwang, P. M., Targeted disruption of p53 attenuates doxorubicin-induced cardiac toxicity in mice, Mol. Cell. Biochem., 2005, 273, 25-32
Shnevvays, V. Mamedova, L. Zinman, T. Jacobson, K. Shainberg, A., Activation of A₃adenosine receptor protects against doxorubicin-induced cardiotoxicity, J. Mol. Cell. Cardiol., 2001, 33, 1249-1261
Simpson, L. L., Evidence that deprenyl, a type B monoamine oxidase inhibitor is an indirectly acting sympathomimetic amine, Biochem. Pharmacol., 1978, 27, 1591-1595
Spallarossa, P. Garibaldi, S. Altieri, P. Fabbi, P. Manca, V. Nasti, S. Rossettin, P. Ghigliotti, G. Ballestrero, A. Patrone, F. Barsotti, A. Brunelli, C., Carvedilol prevents doxorubicin-induced free radical release and apoptosis in cardiomyocytes in vitro, J. Mol. Cell. Cardiol., 2004, 37, 837-846
Swain, S. M. Whaley. F. S. Ewer, M. S., Congestive heart failure in patients treated with doxorubicin: a retrospective analysis of three trials, Cancer, 2003, 97, 2869-2879
Szoko, E. Kalasz, H. Magyat, K., Biotransformation of deprenyl enantiomers, Eur. J. Drug. Metab. Pharmacokinet., 1999, 24, 315-319
Tatton, W. G., Selegiline can mediate neuronal rescue rather than neuronal protection. Movement Disorders 8 (Supp. 1), 1993, S20-S30
Tatton W. G. Greenwood, C. E., Rescue of dying neurons: a new action for deprenyl in MPTP parkinsonism, J. Neurosci. Res., 1991, 30(4), 666-672
Timolati, F. Ott, D. Pentassuglia, L. Giraud, M. N. Perriard, J. C. Suter, T. M. Zuppinger, C., Neuregulin-1 beta attenuates doxorubicin-induced alterations of excitation-contraction coupling and reduces oxidative stress in adult rat cardiomyocytes, J. Mol. Cell. Cardiol., 2006, 41, 845-854
Ueno, M. Kakinuma, Y. Yuhki, K. Murakoshi, N. Iemitsu, M. Miyauchi. T. Yamaguchi, I., Doxorubicin induces apoptosis by activation of caspase-3 in cultured cardiomyocytes in vitro and rat cardiac ventricles in vivo. J. Pharmacol. Sci., 2006, 101, 151-158
Wang, G. X. Wang, Y. X. Zhou, X. B. Korth, M., Effects of doxorubicinol on excitation—contraction coupling in guinea pig ventricular myocytes, Eur. J. Pharmacol., 2001, 423, 99-107
Weinreb, O. Bar-Am, O. Amit, T. Chillag-Talmor, O. Youdim, M. B. H., Neuroprotection via pro-survival protein kinase C isoforms associated with Bcl-2 family members. FASEB J, 2004, 18, 1471-1473
Wu, S. Ko, Y. S. Teng. M. S. Ko, Y. L. Hsu, L. A. Hsueh, C. Chou, Y. Y. Liew, C. C. Lee, Y. S., Adriamycin-induced cardiomyocyte and endothelial cell apoptosis: in vitro and in vivo studies, J. Mol. Cell. Cardiol., 2002, 34, 1595-1607
Yaniv, G. Shilkrut, M. Lotan, R. Larish, S. Berke, G. Binah, O., Hypoxia predisposes neonatal rat ventricular myocytes to apoptosis induced by activation of the Fas (CD95/Apo-1) receptor: Fas activation and apoptosis in hypoxic myocytes, Cardiovasc. Res., 2002, 54, 611-623
Yi, H. Maruyama, W. Akao, Y. Takahashi, T. Iwasa, K. Youdiim, M. B. H. Naoi, M., N-Propargylamine protects SH-SY5Y cells from apoptosis induced by an endogenous neurotoxin, N-methyl(R)salsolinol, through stabilization of mitochondrial membrane and induction of anti-apoptotic Bcl-2, J. Neural. Transm. 2006, 113, 21-32
Youdim, M. B. H. Weinstock, M., Molecular basis of neuroprotective activities of rasagiline and the anti-Alzheimer drug TV3326 [(N-propargyl-(3R)aminoindan-5-YL)-ethyl methyl carbamate], Cell. Mol. Neurobiol., 2001, 21, 555-573
Youdim, M. B. H. Gross, A. Finberg, J. P., Rasagiline [N-propargyl-1R(+)-aminoindan], a selective and potent inhibitor of mitochondrial monoamine oxidase B, Br. J. Pharmacol., 2001a, 132, 500-506
Youdim, M. B. H. Wadia, A. Tatton, W. Weinstock, M., The anti-Parkinson drug rasagiline and its cholinesterase inhibitor derivatives exert neuroprotection unrelated to MAO inhibition in cell culture and in vivo, Ann. N Y Acad. Sci., 2001b, 939, 450-458
Youdim, M. B. H., Rasagiline: an antiparkinson drug with neutroprotective activity, Expert. Rev., Neurotherapeutics, 2003, 3, 737-749
Youdim, M. B. H. Amit, T. Falach-Yogev, M. Am, O. B. Maruyama, W. Naoi, M., The essentiality of Bcl-2, PKC and proteasome-ubiquitin complex activations in the neuroprotective-antiapoptotic action of the anti-Parkinson drug, rasagiline, Biochem. Pharmacol., 2003, 66, 1635-1641
Youdim, M. B. H. Bar Am, O. Yogev-Falach, M. Weinreb, O. Maruyama, W. Naoi, M. Amit, T., Rasagiline: neurodegeneration, neuroprotection, and mitochondrial permeability transition, J. Neurosci. Res., 2005, 79, 172-179
Zimmermann, K. Waldmeier, P. C. Tatton, W. G., Dibenzoxepines as treatments for neurodegenerative diseases, Pure Appl Chem, 1999, 71(11), 2039-2046

1. A method for preventing or attenuating anthracycline-induced cardiotoxicity in a patient treated with an anthracycline chemotherapeutic agent, comprising administering to the subject an amount of an active agent selected from the group consisting of propargylamine, a propargylamine derivative, and a pharmaceutically acceptable salt thereof, effective to treat the subject.

2. The method of claim 1, for preventing anthracycline-induced cardiotoxicity in a patient treated with an anthracycline chemotherapeutic agent.

3. The method of claim 1, for attenuating anthracycline-induced cardiotoxicity in a patient treated with an anthracycline chemotherapeutic agent.

4. The method of claim 1, wherein said anthracycline chemotherapeutic agent is selected from the group consisting of daunorubicin, doxorubicin, epirubicin, idarubicin and mitoxantrone.

5. The method of claim 4, wherein said anthracycline chemotherapeutic agent is doxorubicin.

6. The method of claim 1, wherein said anthracycline-induced cardiotoxicity is an acute anthracycline-induced cardiotoxicity.

7. The method of claim 1, wherein said anthracycline-induced cardiotoxicity is a chronic anthracycline-induced cardiotoxicity.

8. The method of claim 1, wherein said active agent is selected from the group consisting of N-propargyl-1-aminoindan, all enantiomer thereof, an analog thereof and a pharmaceutically acceptable salt of the aforesaid.

9. The method of claim 8, wherein said active agent is racemic N-propargyl-1-aminoindan.

10. The method of claim 8, wherein said active agent is the enantiomer R(+) -N-propargyl-1-aminoindan.

11. The method of claim 8, wherein said active agent is the enantiomer S-(−) -N-propargyl-1-aminoindan.

12. The method of claim 8, wherein said active agent is a pharmaceutically acceptable salt of R(+)-N-propargyl-1-aminoindan or S(−)-N-propargyl-1-aminoindan.

13. The method of claim 12, wherein said pharmaceutically acceptable salt is selected from the group consisting of the mesylate salt; the esylate salt; the sulfate salt; and the hydrochloride salt of R(+)-N-propargyl-1-aminoindan or S(−)-N-propargyl-1-aminoindan.

14. The method of claim 8, wherein said analog of N-propargyl-1-aminoindan is selected from the group consisting of 4-fluoro-N-propargyl-1-aminoindan, 5-fluoro-N-propargyl-1-aminoindan, 6-fluoro-N-propargyl-1-aminoindan, an enantiomer thereof and pharmaceutically acceptable addition salts thereof.

15. The method of claim 8, wherein said analog of N-propargyl-1-aminoindan is selected from the group consisting of (rac)-3-(N-methyl,N-propyl-carbamyloxy)-α-methyl-N′-propargyl phenethylamine HCl; (rac)-3-(N,N-dimethyl-carbamyloxy)-α-methyl-N′-methyl, N′-propargyl phenethylamine HCl; (rac)-3-(N-methyl,N-hexyl-carbamyloxy)-α-methyl-N′-methyl. N′-propargyl phenethylamine mesylate; (rac)-3-(N-methyl,N-cyclohexyl-carbamyloxy)-α-methyl-N′-methyl,N′-propargyl phenethylamine HCl; and (S)-3-(N-methyl, N-hexyl-carbamyloxy)-α-methyl-N′-methyl,N′-propargyl phenethylamine ethanesulfonate.

16. The method of claim 8, wherein said analog of N-propargyl-1-aminoindan is selected from the group consisting of (rac) 6-(N-methyl, N-ethyl-carbamyloxy)-N′-propargyl-1-aminoindan HCl; (rac) 6-(N(N-dimethyl, carbamyloxy)-N′-methyl-N′-propargyl-1-aminoindan HCl (rac) 6-(N-methyl, N-ethyl-carbamyloxy-N′-propargyl-1-aminotetralin HCl; (rac) 6-(N,N-dimethyl-thiocarbamyloxy)-1-aminoindan HCl; (rac) 6-(N-propyl-carbamyloxy-N′-propargyl-1-aminoindan HCl; (rac) 5-chloro-6-(N-methyl, N-propyl-carbamyloxy)-N′-propargyl-1-aminoindan HCl; (S)-6-(N-methyl), N-propyl-carbamyloxy)-N′-propargyl-1-aminoindan HCl; and (R)-6-(N-methyl, N-ethyl-carbamyloxy)-N′-propargyl-1-aminoindan hemi-(L)-tartrate. 6 and 6-(N-methyl, N-ethyl-carbamyloxy)-N-methyl,N′-propargyl-1-aminoindan.

17. The method of claim 1, wherein said active agent is an aliphatic propargylamine.

18. The method of claim 17, wherein said aliphatic propargylamine is selected from the group consisting of the compounds N-(1-heptyl)propargylamine or a propargylamine; N-(1-octyl)propargylamine; N-(1-nonyl)propargylamine; N-(1-decyl)propargyl-amine; N-(1-undecyl)propargylamine; N-(1-dodecyl) propargylamine; R—N-(2-butyl)propargylamine; R—N-(2-pentyl) propargylamine; R—N-(2-hexyl) propargylamine; R—N-(2-heptyl)propargylamine; R—N-(2-octyl) propargylamine; R—N-(2-nonyl)propargylamine; R—N-(2-decyl) propargylamine, R—N-(2-undecyl) propargylamine; R—N-(2-dodecyl)propargylamine; N-(1-butyl)-N-methylpropargyl-amine; N-(2-butyl)-N-methylpropargylamine; N-(2-pentyl)-N-methylpropargyl-amine; N-(1-pentyl)-N-methylpropargylamine; N-(2-hexyl)-N-methylpropargyl-amine; N-(2-heptyl)-N-methylpropargylamine; N-(2-decyl)-N-methylpropargyl-amine; N-(2-dodecyl)-N-methylpropargylamine; R(−)-N-(2-butyl)-N-methyl-propargylamine; or a pharmaceutically acceptable salt thereof.

19. The method of claim 1, wherein said active agent is selected from the group consisting of selegiline, desmethylselegiline, pargyline, chlorgyline and N-methyl-N-propargyl-10-aminomethyl-dibenzo[b,f]oxepin.

20. The method of claim 1, wherein said active agent is propargylamine or a pharmaceutically acceptable salt thereof.
